(19) United States
(12) Patent Application Publication
(10) Pub. No.: US 2015/0017658 A1
(43) Pub. Date: Jan. 15, 2015

(54) STAIN, PROCESS FOR STAINING AND ACQUIRING NORMALIZED SIGNAL

(71) Applicant: NATIONAL INSTITUTE OF STANDARDS AND TECHNOLOGY, GAITHERSBURG, MD (US)

(72) Inventors: GREGORY ALAN COOKSEY, GAITHERSBURG, MD (US); JOHN T. ELLIOTT, GAITHERSBURG, MD (US); ANNE L. PLANT, WASHINGTON, DC (US)

(21) Appl. No.: 14/499,364

(22) Filed: Sep. 29, 2014

Related U.S. Application Data
(60) Provisional application No. 62/000,107, filed on May 19, 2014.

(51) Int. Cl. G01N 33/569; G01N 33/533; G01N 33/535

(52) U.S. Cl. CPC G01N 33/56966; G01N 33/535; G01N 33/533; G01N 2496/00
USPC 435/7.9; 435/7.1

(57) ABSTRACT

A kit includes a reference probe to react with a first sample; and an analyte probe to combine with a second sample, a marker and a spectral probe to react to form the analyte probe to combine with the second sample, the marker to react with the reference probe to form the analyte probe to combine with the second sample, or a combination thereof. A process for staining includes forming a reference composition by reacting a reference probe and a first sample to form a reference; and forming an analyte composition by combining an analyte probe and a second sample to form an analyte.

### CROSS REFERENCE TO RELATED APPLICATIONS

This application claims the benefit of U.S. Provisional Patent Application Ser. No. 62/000,107 filed May 19, 2014, the disclosure of which is incorporated herein by reference in its entirety.

### STATEMENT REGARDING FEDERALLY SPONSORED RESEARCH

This invention was made with United States government support from the National Institute of Standards and Technology. The government has certain rights in the invention.

### BACKGROUND

A reference material such as fluorescent beads or glasses are used to normalize data from day-to-day or lab-to-lab investigations that involve fluorescence measurements. However, properties of an instrument or the reference material make comparison of fluorescence data difficult or impractical, particularly for fluorescence data from different laboratories or instruments or among fluorescence data from the same lab acquired on different days. Limitations include spectral differences between the reference material and a fluorophore as well as optical properties of instrumentation such as alignment, temporal stability, detector drift, and the like.

Accordingly, the art is receptive to materials and methods that overcome such difficulties.

### BRIEF DESCRIPTION

The above and other deficiencies are overcome by, in an embodiment, a kit comprising: a reference probe to react with a first sample; and an analyte probe to combine with a second sample, a marker and a spectral probe to react to form the analyte probe to combine with the second sample, the marker to react with the reference probe to form the analyte probe to combine with the second sample, or a combination comprising at least one of the foregoing.

Further disclosed is a process for staining, the process comprising: forming a reference composition by reacting a reference probe and a first sample to form a reference; and forming an analyte composition by combining an analyte probe and a second sample to form an analyte.

Additionally disclosed is a process for obtaining a normalized signal from an analyte, the process comprising: combining a reference probe and a first sample to produce a reference composition; reacting the reference probe and the first sample to form a reference in the reference composition; determining a reference signal from the reference in response to subjecting the reference to radiation; combining an analyte probe and a second sample to produce an analyte composition; contacting the analyte probe and the second sample to form an analyte in the second composition; determining an analyte signal from the analyte in response to subjecting the analyte to radiation; and obtaining the normalized signal by normalizing the analyte signal to the reference signal.

### BRIEF DESCRIPTION OF THE DRAWINGS

The following descriptions should not be considered limiting in any way. With reference to the accompanying drawings, like elements are numbered alike:

FIG. 1 shows a kit;

FIG. 2 shows a kit;

FIG. 3 shows a kit;

FIG. 4 shows a kit;

FIG. 5 shows a kit;

FIG. 6 shows a flow chart for normalizing analyte signal;

FIG. 7 shows a graph of intensity versus wavelength;

FIG. 8 shows a graph of signal versus item;

FIG. 9 shows a graph of normalized signal versus item;

FIG. 10 shows a flow chart for normalizing analyte signal;

FIG. 11 shows a graph of intensity versus wavelength;

FIG. 12 shows a graph of signal versus item; and

FIG. 13 shows a graph of normalized signal versus item.

### DETAILED DESCRIPTION

A detailed description of one or more embodiments is presented herein by way of exemplification and not limitation.

It has been found that a reference and an analyte system provide comparability of biomarker measurements between cell samples and across laboratories. The reference and analyte advantageously are applied to microscopy and flow cytometry. Beneficially, a process for staining and obtaining the normalized signal therefrom is near-universal and instrument independent. As such, articles and processes herein provide a comparison of different diagnostic and therapeutic products and facilitation of design and evaluation of biologic materials. The reference process provides for staining and obtaining a normalized signal that is substantially insensitive to concentrations and staining times. Total cellular protein is obtained using the reference, the process being a benchmark upon which an analyte is compared ratiometrically.

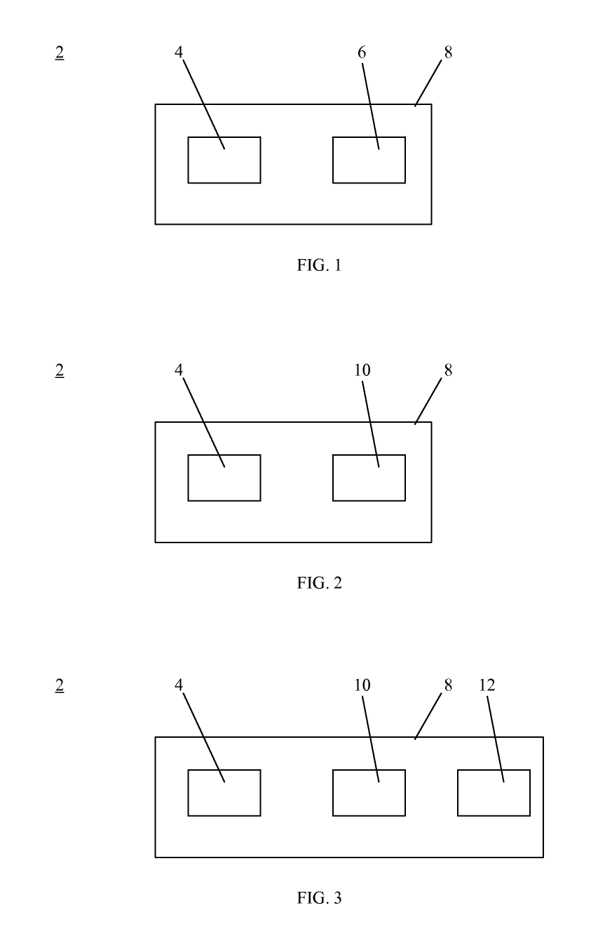

According to an embodiment, as shown in FIG. 1, kit 2 includes reference probe 4 and analyte probe 6 that are optionally disposed in container 8. Reference probe 4 is provided to react with a first sample to form a reference. Analyte probe 6 is provided to combine with a second sample to form an analyte.

In an embodiment, as shown in FIG. 2, kit 2 includes reference probe 4 and marker 10 optionally disposed in container 8. Reference probe 4 is provided to react with the first sample to form the reference. Marker 10 is provided to react with reference probe 4 to form the analyte probe, which would be provided to combine with the second sample to form the analyte.

In an embodiment, as shown in FIG. 3, kit 2 includes reference probe 4, marker 10, and spectral probe 12 optionally disposed in container 8. Here, marker 10 is provided to react with spectral probe 12 to form the analyte probe, which would combine with the second sample to form the analyte. In some embodiments, marker 8 is provided to react with reference probe 4, spectral probe 12, or a combination thereof to form the analyte probe, which would combine with the second sample to form the analyte.

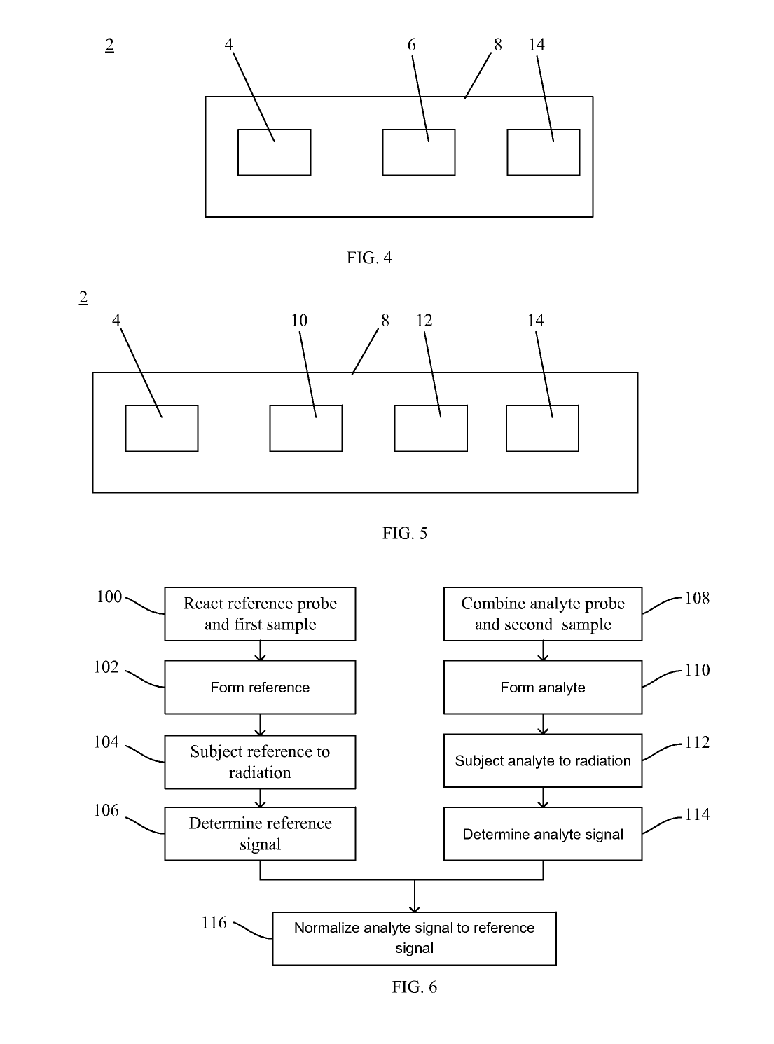

According to an embodiment, as shown in FIG. 4, kit 2 includes reference probe 4, analyte probe 6, and calibration probe 14 optionally disposed in container 8. Reference probe 4 is provided to react with the first sample to form the reference. Analyte probe 6 is provided to combine with the second sample to form the analyte. According to an embodiment, calibration probe 14 is provided to react with the second sample to form a first calibrant. In some embodiments, calibration probe 14 is provided to react with a third sample to form a second calibrant. In a particular embodiment, calibration probe 14 is provided to react with the second sample to form the first calibrant, the third sample to form the second calibrant, or combination thereof.

In an embodiment, as shown in FIG. 5, kit 2 includes reference probe 4 to react with the first sample; and analyte probe 6 to combine with the second sample, marker 10 and spectral probe 12 to react to form analyte probe 6 to combine with the second sample, marker 10 to react with reference probe 4 to form analyte probe 6 to combine with the second sample, calibration probe 14, or a combination comprising at least one of the foregoing.

In some embodiments, kit 2 includes a permeation agent, a reducing agent, a supplemental agent, a buffering agent, or combination thereof.

Reference probe 4 reacts with the first sample and includes a compound of formula 1, formula 2, formula 3, or a combination thereof.

Q1-L1-A1 (1)

Q1-A1 (2)

Q1-R (3)

wherein Q1 is a moiety that is reactive with an amino acid residue, peptide, nucleic acid, and the like; L1 is a linker group; A1 is a spectrally active group; and R is a terminal group.

Reactive moiety Q1 is a chemical group that reacts with an amino acid, protein, peptide, nucleic acid, and the like. A variety of reactive moieties Q1 are disclosed in the following sources: The Molecular Probes Handbook, 11th Ed., Cross-linking and Photoactivatable Reagents, Chapter 5 (Invitrogen Life Science); Bioconjugate Reagents, Bioconjugate Techniques, Part I and II, 2nd Ed, by Greg T. Hermanson, (Academic Press 2008); and Photoreactive Crosslinking and Labeling Reagents, Crosslinking and Photoreactive Reagents, Chapter 5, (Molecular Biotechnology, MoBiTech), the contents of each of these references are herein incorporated by reference in their entirety.

Some non-limiting examples of reactive moiety Q1 include: N-hydroxysuccinimide (NHS) esters (amine reactive), N-hydroxysulfosuccinimide (sulfo-NHS) esters (amine reactive), succinimidyl acetylthioacetate (SATA), carbodiimides (amine and carboxyl reactive), hydroxymethyl phosphines (amine reactive), maleimides (thiol reactive), aryl azides (primary amine reactive), fluorinated aryl azides (carbon-hydrogen (C—H) insertion), pentafluorophenyl (PFP) esters (amine reactive), imidoesters (amine reactive), isocyanates (hydroxyl reactive), psoralen (a photoreactive intercalator that reacts with thymine), vinyl sulfones (reacts with thiols, amines, and hydroxyls), pyridyl disulfides (reacts with thiols), benzophenone derivatives (C—H bond insertion), and the like.

In an embodiment, Q1 is an amine reactive moiety, a thiol reactive moiety, or combination thereof. As used herein, “amine reactive moiety” means that group Q1 reacts with an amine group. As used herein, “thiol reactive moiety” means that group Q1 reacts with a thiol group. Furthermore, a thiol (or thiol group) is often referred to as a sulfhydryl (sulfhydryl group). Exemplary amine reactive moieties Q1 include an acetimidate, acyl azide, aldehyde, anhydride, aryl halide, carbodiimide, carbonate, epoxide, fluorophenyl ester, glyoxal, imidoester, isocyanate, isothiocyanate, N-hydroxysuccinimide ester (NHS ester), sulfonyl chloride, sulfo-N-hydroxysuccinimide ester (sulfo-NHS ester; having a structure identical to NHS esters but containing a sulfonate group on the N-hydroxysuccinimide ring), halogenated dinitrobenzene, and the like having respective structures as follows:

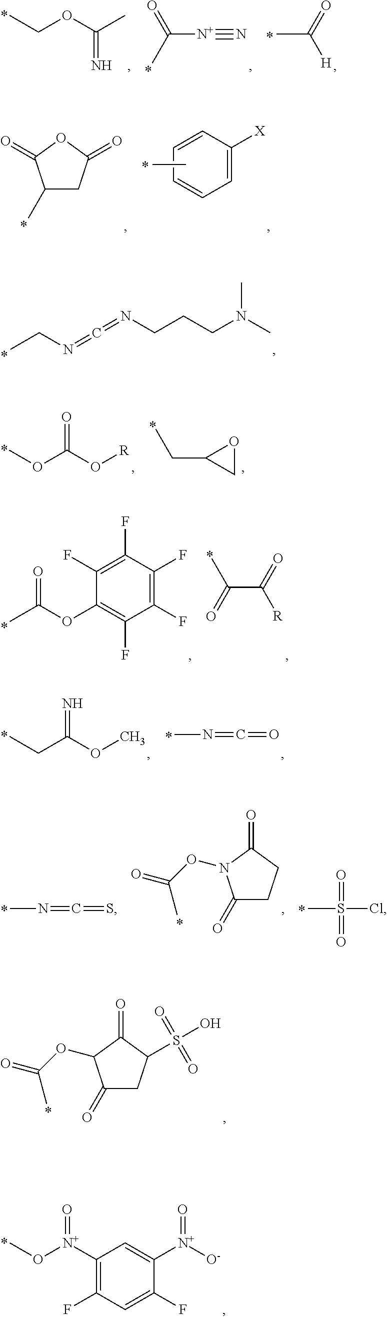

and the like, wherein asterisk (*) represents a point of attachment in reactive moiety Q1 of reference probe 4; X is halogen (e.g., Cl, F, Br, I, and the like); and R is a terminal group.

Exemplary terminal groups R include H, F, Cl, Br, I, OH, SH, NHOH, NHNH2, CHO, C(═O)OH, alkenyl, alkoxy, alkyl, alkylamine, alkylaryl, alkynyl, amide, amine, amino, aralkyloxy, aryl, arylalkyl, aryleneamine, aryloxy, carbocyclic, carboxylic acid group or salt thereof, cycloalkyl, cycloalkyloxy, cycloalkenyl, cycloalkynyl, haloalkyl, heteroaralkyl, heteroaryl, or heterocycloalkyl.

Exemplary thiol reactive moieties Q1 include a maleimide, vinyl sulfone, pyridyl disulfide, dinitrobenzenesulfonyl group, and the like having respective structures:

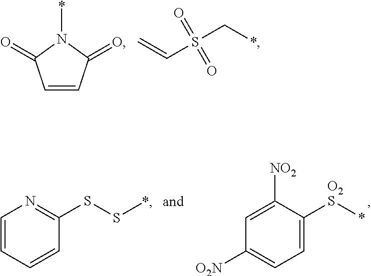

wherein asterisk (*) is a point of attachment.

Linker L1 is a group that links reactive moiety Q1 to spectrally active group A1 in reference probe 4. In some embodiments, linker L1 is a linkage comprising 1 to 20 carbon atoms, or 1 to 6 polyethylene glycol groups. In some embodiments, linker L1 is selected to obtain a particular length of reference probe 4 according to the first sample with which reference probe 4 reacts. As such, a length of linker L1 is tunable.

Exemplary linkers L1 include an element selected from group 13, 14, 15, 16 of the periodic table (e.g., O, S, N, B, C), a poly atomic divalent group such as R¹, OR¹, NR¹, CR¹₂, CR¹₂CR¹₂, CR¹₂O, CR¹₂OCR¹₂, CR¹₂S, CR¹₂SCR¹₂, CR¹₂NR¹, CR¹₂NR¹CR¹₂, alkenylene, alkylene, alkyleneoxy, alkynylene, amide, amine, aralkylene, arylene, aryleneoxy, cycloalkylene, fluoroalkylene, heteroaralkylene, heteroarylene, heterocycloalkylene, a single bond, and the like.

Exemplary R¹ groups for linker L1 include —O—, —NH—, —S—, —C(O)—, C(O)—NH, NH—C(O)—NH, O—C(O)—NH, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂— CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂, —CH₂—CH₂—NH—C(O)—CH₂—CH₂, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, and the like.

Spectrally active group A1 is a fluorescent, luminescent, phosphorescent, chemiluminescent, or chromagenic moiety. Spectrally active groups A1 that may be used include a variety of organic or inorganic small molecules commonly referred to as a dye, label, or indicator that absorb or emit a wavelength the electromagnetic spectrum, e.g., ultraviolet, visible, near infrared, or infrared wavelengths. Spectrally active groups A1, e.g., include organic dyes, inorganic phosphors, semiconducting nanocrystals, and the like. In an embodiment, spectrally active group A1 is a bodipy dye, perylene, pyromethene, rhodamine, sulforhodamine, coumarin, aluminum quinoline complex, porphyrin, porphin, indocyanine dye, phenoxazine derivative, phthalocyanine dye, polymethyl indolium dye, polymethine dye, guaiazulenyl dye, croconium dye, polymethine indolium dye, metal complex IR dye, cyanine dye, squarylium dye, chalcogeno-pyryloarylidene dye, indolizine dye, pyrylium dye, quinoid dye, quinone dye, azo dye, derivatives thereof, or a combination thereof. Exemplary porphyrin and porphyrin derivatives include those available from Frontier Scientific such as etioporphyrin 1 (CAS #448-71-5), deuteroporphyrin IX 2,4 bis ethylene glycol (part #D630-9), and octaethyl porphrin (CAS 2683-82-1); azo dyes such as mordant orange (CAS #2243-76-7), methyl yellow (CAS #60-11-7), 4-phenylazoaniline (CAS #60-09-3), alcian yellow (CAS #61968-76-1), available from Aldrich Chemical Company, and the like.

Additional exemplary spectrally active groups A1 include a fluorescein, a rhodamine, an oxazine, an acridine dye, a cyanine dye, and the like, particularly fluorescein diphosphate (tetraammonium salt), fluorescein 3′(6′)-O-alkyl-6′(3′)-phosphate, 9H-(1,3-dichloro-9,9-dimethylacridin-2-one-7-yl)phosphate (diammonium salt), 4-methylumbelliferyl phosphate, resorufin phosphate, 4-trifluoromethylumbelliferyl phosphate, umbelliferyl phosphate, 3-cyanoubelliferyl phosphate, 9,9-dimethylacridin-2-one-7-yl phosphate, 6,8-difluoro-4-methylumbelliferyl phosphate, and derivatives thereof, and the like.

In a particular embodiment, spectrally active group A1 has a structure as follows:

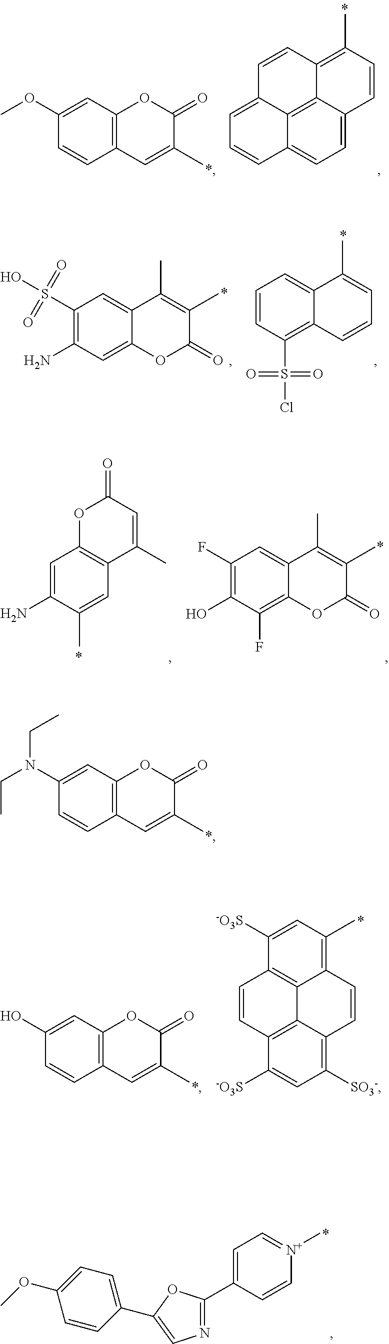

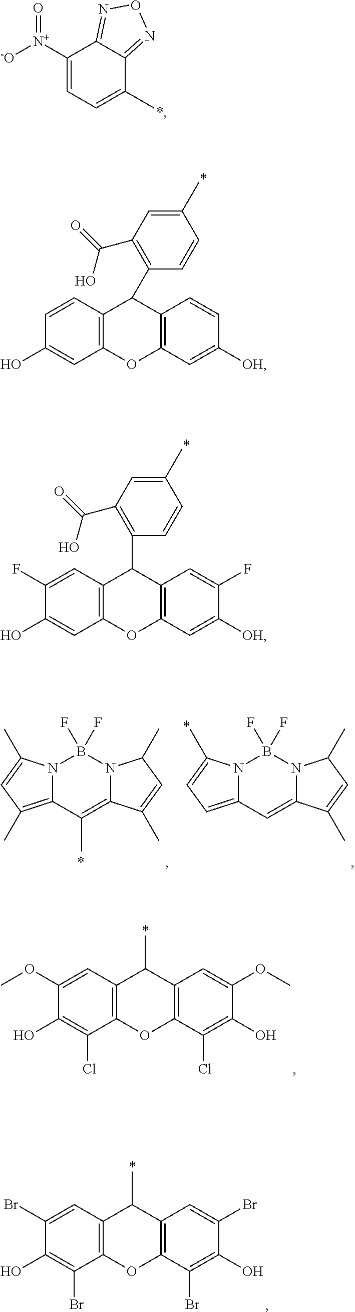

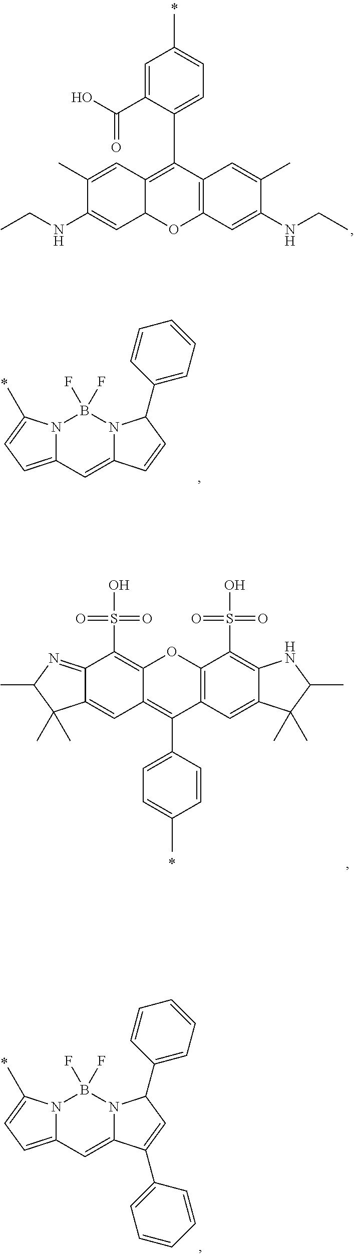

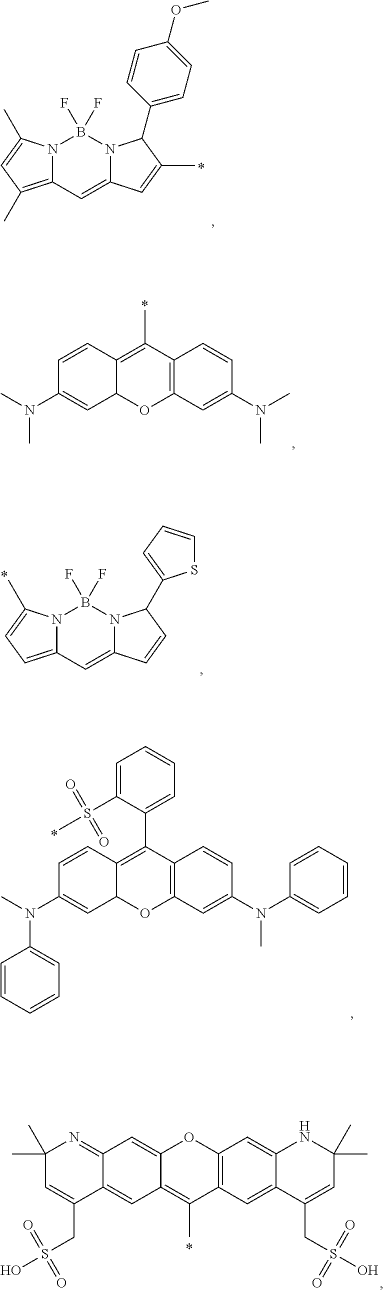

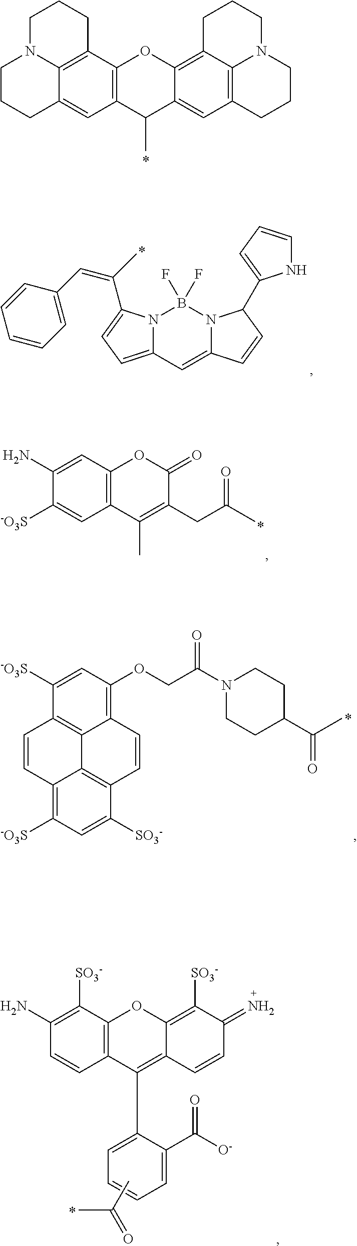

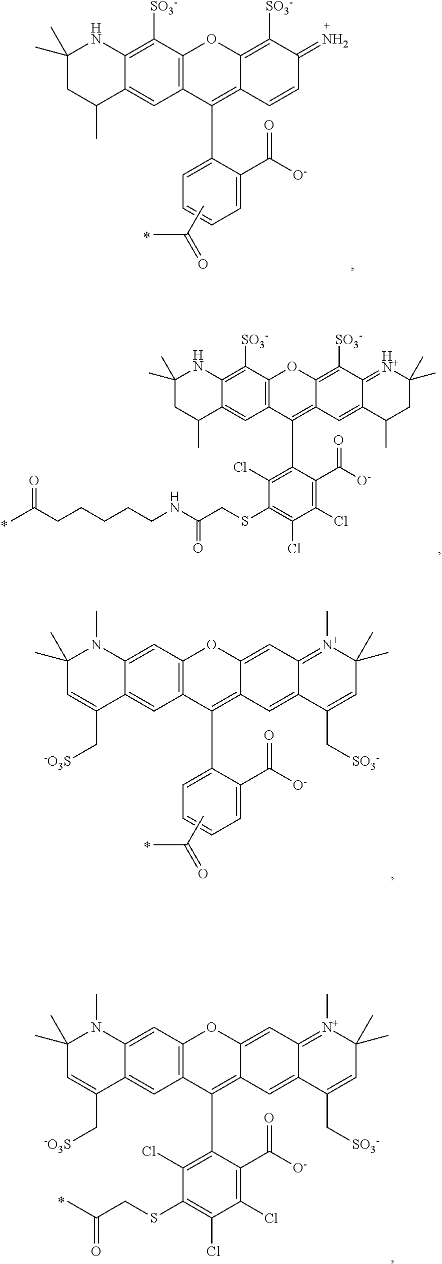

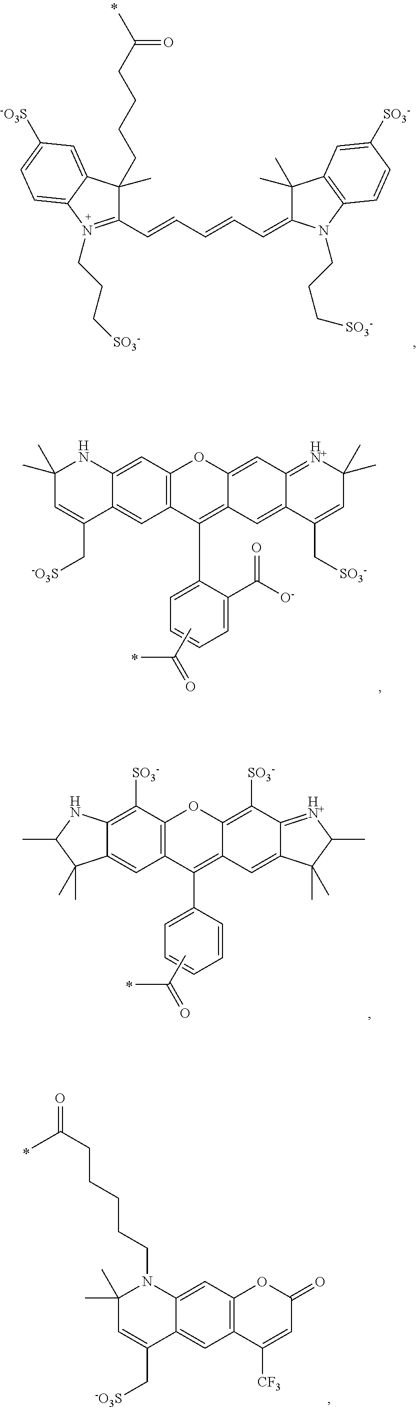

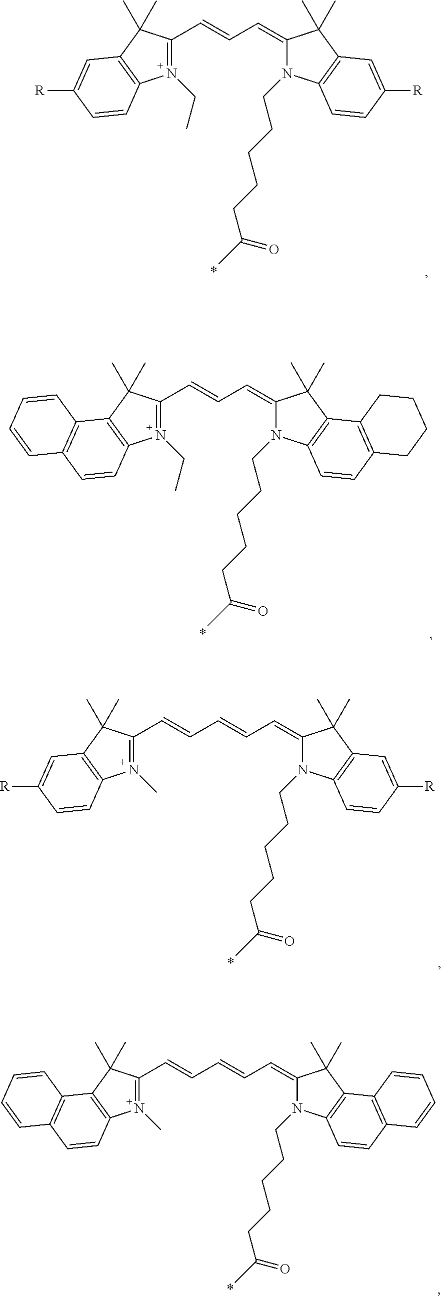

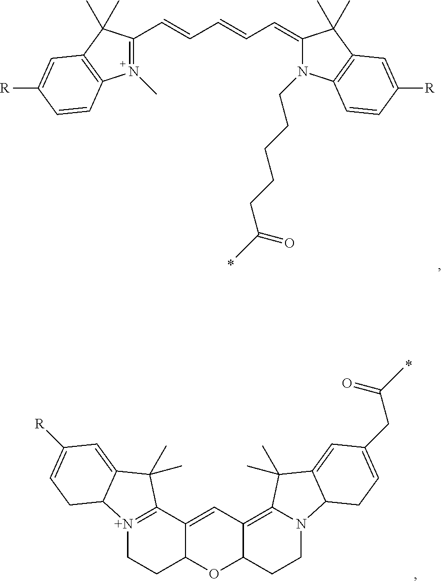

and the like, or combination thereof, wherein R is a group as described above, and asterisk # is a point of attachment.

Analyte probe 6 combines with the second sample and includes a compound of formulas 4a, 4b, 4c, 5a, 5b, 6a, 6b, or a combination thereof.

M-Q′-L1-A1 (4a)

M-Q′-A1 (4b)

M-Q′-A1-R (4c)

M-A1 (5a)

M-A1-R (5b)

M-L1-A1 (6a)

M-L1-A1-R (6c)

wherein, as described for reference probe 4, Q′ is a product moiety (e.g., a moiety that is derived from reaction between reactive moiety Q1 and marker M); L1 is a linker group; A1 is a spectrally active group; R is a terminal group; and wherein M is a marker. Exemplary product moieties Q′ have a structure such as

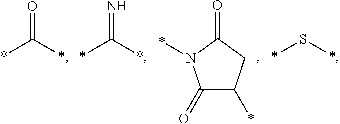

and the like.

According to an embodiment, analyte probe 6 and reference probe 4 have a same or different linker L1, a same or different spectrally active A1, or a same or different terminal group R. In an embodiment, L1 and A1 respectively are identical in analyte probe 6 and reference probe 4. In a particular embodiment, spectrally active group A1 is the same in analyte probe 6 and reference probe 4 but L1 is different in analyte probe 6 and reference probe 4.

Analyte probe 6 includes marker M (e.g., in formulas 4a, 4b, 4c, 5a, 5b, 6a, or 6b). In an embodiment, analyte probe 6 contacts the second sample so that marker M portion of analyte probe 6 combines with the second sample and forms the analyte. In an embodiment, marker M reacts with the second sample, binds to the second sample, electrically associates with the second sample, or a combination thereof. In some embodiments, analyte probe 6 is formed from marker M. Here, marker M is included in kit 2 to react with reference probe 4 to form analyte probe 6. That is, marker M and reactive moiety Q1 of reference probe 4 react to form analyte probe 6, e.g., as follows:

M (marker)+Q1-L1-A1 (reference probe 4)→M-Q′-L1-A1 (analyte probe 6).

In an embodiment, where analyte probe 6 is formed in such a reaction between marker M and reference probe 4, a structure of product moiety Q′ depends on a structure of reactive moiety Q1 as well as a chemical makeup of marker M. In a particular embodiment, Q1 is an amino reactive moiety in reference probe 4, and marker M includes an amino group such that Q′ includes a carbonyl group or imine group. In a certain embodiment, Q1 is a thiol reactive moiety in reference probe 4, and marker M includes a thiol group such that Q′ includes, e.g., a divalent sulfur (*—S—*) group, 1λ²,3λ³-pyrrolidine-2,5-dione group, and the like.

According to an embodiment, the kit includes analyte probe 6 or optionally marker M. Exemplary markers M include an antibody, antibody mimetic, amino acid, and the like. In an embodiment, marker M interacts with an analyte such that marker M is a pharmacological agent that binds the analyte with high affinity or high specificity. It is contemplated that marker M is a small molecule, peptide, protein, and the like. In an embodiment, marker M is a toxin, e.g. phalloidin, tetrodotoxin, and the like, that can be fluorescently labeled to form an analyte probe.

The antibody can be an immunoglobulin molecule or an immunologically active portion of an immunoglobulin molecule (i.e., a molecule that contains an antigen binding site that binds an antigen), synthetic antibody, and the like. The immunoglobulin molecule can be of any class (e.g., IgG, IgE, IgM, IgD IgA, IgY, IgW, and the like) or subclass of immunoglobulin molecule. The antibody includes, e.g., a polyclonal, monoclonal, bispecific, synthetic, humanized or chimeric antibody, single chain antibody, Fab fragment and F(ab′)2 fragment, Fv or Fv′ portion, fragment produced by a Fab expression library, anti-idiotypic (anti-Id) antibody, or epitope-binding fragment of any of the above, and the like. According to an embodiment, an antibody, or generally any molecule, “binds specifically” to an antigen (or other molecule) if the antibody binds preferentially to the antigen, and, e.g., has less than or equal to 30%, specifically less than or equal to 20%, more specifically less than or equal to 10%, further specifically less than or equal to 5%, and additionally specifically less than or equal to 1% cross-reactivity with another molecule.

The term “mimetic” as used herein refers to any entity, including natural and synthesized inorganic or organic molecules, including recombinant molecules, which mimic the properties of the molecule of which it is a mimetic. Accordingly, a mimetic of a particular antibody has the same, similar or enhanced epitope binding properties of that antibody. Exemplary antibody mimetics include affibody molecules, affilins, affitins, anticalins, avimers, DARPinsm, fynomers, Kunitz domain peptides, monobodies, nucleic acids, and the like.

According to an embodiment, the marker M is a monoclonal antibody such as an anti-inflammatory, anti-cancer, anti-viral monoclonal antibody, and the like. Exemplary therapeutic monoclonal antibodies include types such as infliximab (available under the trade name Remicade), adalimumab (available under the trade name Humara), basiliximab (available under the trade name Simulect), daclizumab (available under the trade name Zenapax), omalizumab (available under the trade name Xolair), Gemtuzumab (available under the trade name Mylotarg), alemtuzumab (available under the trade names Campath, MabCampath and Campath-1H), rituximab (available under the trade names Rituxan, MabThera, and Zytux), trastuzumab (available under the trade names Herclon, Herceptin), nimotuzumab (CAS #828933-51-3), cetuximab (available under the trade name Erbitux), bevacizumab (available under the trade name Erbitux), bavituximab (CAS #648904-28-3), palivizumab (available under the trade name Erbitux), abciximab (available under the trade name ReoPro), and the like. Further exemplary markers M include a diagnostic antibody that determine a pluripotency or differentiation state of a cell type, e.g., an antibody against Tra-1-60, Tra-1-81, Oct 3/4, Nanog, Sox2, E-cadherin, SSEA1, CD34, and the like.

Additional marker M antibodies include (using nomenclature of monoclonal antibodies as adopted as World Health Organization's International Nonproprietary Names (INN) and as United States Adopted Names (USAN) for pharmaceuticals) 3F8, 8H9, abagovomab, actoxumab, adecatumumab, aducanumab, afelimomab, afutuzumab, alacizumab pegol, ALD518, alirocumab, altumomab pentetate, amatuximab, anatumomab mafenatox, anifrolumab, anrukinzumab, apolizumab, arcitumomab, aselizumab, atinumab, atlizumab, atorolimumab, bapineuzumab, bectumomab, belimumab, benralizumab, bertilimumab, besilesomab, bezlotoxumab, biciromab, bimagrumab, bivatuzumab mertansine, blinatumomab, blosozumab, brentuximab vedotin, briakinumab, brodalumab, canakinumab, cantuzumab mertansine, cantuzumab ravtansine, caplacizumab, capromab pendetide, carlumab, catumaxomab, cBR96-doxorubicin immunoconjugate, CC49, cedelizumab, certolizumab pegol, citatuzumab bogatox, cixutumumab, clazakizumab, clenoliximab, clivatuzumab tetraxetan, conatumumab, concizumab, CR6261, crenezumab, dacetuzumab, dalotuzumab, daratumumab, demcizumab, denosumab, detumomab, dorlimomab aritox, drozitumab, duligotumab, dupilumab, dusigitumab, ecromeximab, eculizumab, edobacomab, edrecolomab, efalizumab, efungumab, eldelumab, elotuzumab, elsilimomab, enavatuzumab, enlimomab pegol, enokizumab, enoticumab, ensituximab, epitumomab cituxetan, epratuzumab, erlizumab, ertumaxomab, etaracizumab, etrolizumab, evolocumab, exbivirumab, fanolesomab, faralimomab, farletuzumab, fasinumab, FBTA05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gevokizumab, girentuximab, glembatumumab vedotin, golimumab, gomiliximab, guselkumab, ibalizumab, ibritumomab tiuxetan, icrucumab, igovomab, IMAB362, imciromab, imgatuzumab, inclacumab, indatuximab ravtansine, inolimomab, inotuzumab ozogamicin, intetumumab, ipilimumab, iratumumab, itolizumab, ixekizumab, keliximab, labetuzumab, lambrolizumab, lampalizumab, lebrikizumab, lemalesomab, lerdelimumab, lexatumumab, libivirumab, ligelizumab, lintuzumab, lirilumab, lodelcizumab, lorvotuzumab mertansine, lucatumumab, lumiliximab, mapatumumab, margetuximab, maslimomab, matuzumab, mavrilimumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nivolumab, nofetumomab merpentan, ocaratuzumab, ocrelizumab, odulimomab, ofatumumab, olaratumab, olokizumab, onartuzumab, ontuxizumab, oportuzumab monatox, oregovomab, orticumab, otelixizumab, otlertuzumab, oxelumab, ozanezumab, ozoralizumab, pagibaximab, panitumumab, pankomab, panobacumab, parsatuzumab, pascolizumab, pateclizumab, patritumab, pemtumomab, perakizumab, pertuzumab, pexelizumab, pidilizumab, pinatuzumab vedotin, pintumomab, placulumab, polatuzumab vedotin, ponezumab, priliximab, pritoxaximab, pritumumab, pro 140, quilizumab, racotumomab, radretumab, rafivirumab, ramucirumab, ranibizumab, raxibacumab, regavirumab, reslizumab, rilotumumab, robatumumab, roledumab, romosozumab, rontalizumab, rovelizumab, ruplizumab, samalizumab, sarilumab, satumomab pendetide, secukinumab, seribantumab, setoxaximab, sevirumab, SGN-CD19A, SGN-CD33A, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, teneliximab, teplizumab, teprotumumab, TGN1412, ticilimumab, tigatuzumab, tildrakizumab, TNX-650, tocilizumab, toralizumab, tositumomab, tovetumab, tralokinumab, TRBS07, tregalizumab, tremelimumab, tucotuzumab celmoleukin, tuvirumab, ublituximab, urelumab, urtoxazumab, ustekinumab, vantictumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox, and the like.

Spectral probe 12 is included in kit 2 to react with marker 10 to produce analyte probe 6. Spectral probe 12 includes a structure as Q1-L1-A1, Q1-A1, Q1-A1-R, A1, A1-R, Z-L1-A1, or Z-L1-A1-R, wherein is a group that completes a valence of the group that Z is attached to, e.g., A1 or L1. Exemplary Z groups include an electron, hydrogen, R, and the like, which may be removed or incorporated into analyte probed 6 that is formed in reaction between spectral probe 12 and marker 10. That is, spectral probe 12 and marker M react to form analyte probe 6, e.g., as follows: M (marker)+Q1-L1-A1 (spectral probe 4)→M-Q′-L1-A1 (analyte probe 6). Here, a structure of product moiety Q′ depends on a structure of reactive moiety Q1 as well as a chemical makeup of marker M. In a particular embodiment, Q1 is an amino reactive moiety in reference probe 4, and marker M includes an amino group such that Q′ includes a carbonyl group or imine group. In a certain embodiment, Q1 is a thiol reactive moiety in reference probe 4, and marker M includes a thiol group such that Q′ includes, e.g., a divalent sulfur (*—S—*) group, 1λ²,3λ³-pyrrolidine-2,5-dione group, and the like. Additionally, in spectral probe 12, Q1, L1, A1, and R are as previously recited. In an embodiment, A1 in spectral probe 12 is the same is A1 in reference probe 4, and Q1, L1, or R are selected independently for spectral probe 12 and reference probe 4.

According to an embodiment, kit 2 includes first calibration probe, second calibration probe, or combination thereof, collectively referred to as calibration probes. The first calibration probe is provided to react with the second sample in the analyte composition to form the first calibrant. The second calibration probe is provided to react with the third sample in the calibration composition to form the second calibrant.

The first calibration probe and the second calibration probe independently have a structure of formula 7, formula 8, formula 9 or combination thereof.

Q2-L2-A2 (7)

Q2-A2 (8)

Q2-R (9)

wherein Q2 is a moiety that is reactive with an amino acid residue, peptide, nucleic acid, and the like; L2 is a linker group; A2 is a spectrally active group; and R is a terminal group.

In an embodiment, the first calibration probe is the same as the second calibration probe such that the first calibration probe and the second calibration probe include the same reactive moiety Q2. Accordingly, in some embodiments, the first calibration probe and the second calibration probe have a substantially same fluorescence spectrum. According to an embodiment, the first calibration probe includes reactive moiety Q2 that is different than reactive moiety Q2 included in the second calibration probe such that the first calibration probe and the second calibration probe have different fluorescence spectra.

In an embodiment, Q2 is an amine reactive moiety, a thiol reactive moiety, or combination comprising at least one of the foregoing. According to an embodiment, Q2 is a group previously as disclosed for reactive moiety Q1 with regard to analyte probe 6 and reference probe 4. In an embodiment, Q2 of the calibration probes is different than Q1 of reference probe 4 or Q1 of analyte probe 6. In a certain embodiment, Q2 of the calibration probes is the same as Q1 of reference probe 4 or Q1 of analyte probe 6.

According to an embodiment, linker L2 of the calibration probes is a group previously disclosed for linker L1 of reference probe 4 or analyte probe 6. Linker L2 can be a same or different group than linker L1.

According to an embodiment, spectrally active group A2 of the calibration probes is a group previously disclosed for spectrally active group A1 of reference probe 4 or analyte probe 6. Spectrally active group A2 can be a same or different group than spectrally active group A1.

According to an embodiment, terminal group R of the calibration probes is a group previously disclosed for terminal group R of reference probe 4 or analyte probe 6. Terminal group R of the calibration probes can be a same or different than terminal group R of reference probe 4 or analyte probe 6.

Exemplary probes (e.g., reference probe 4, first calibration probe, second calibration probe) include

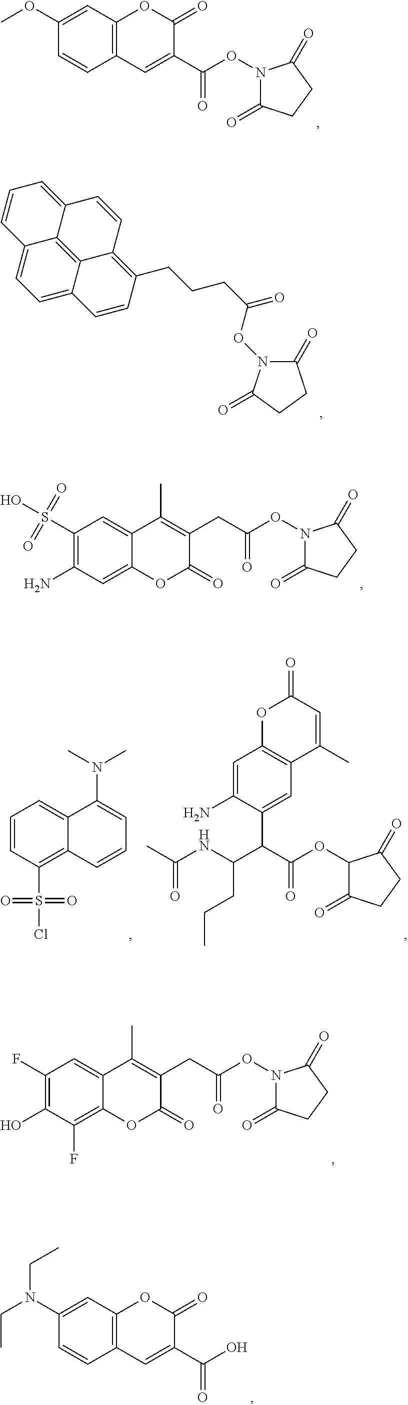

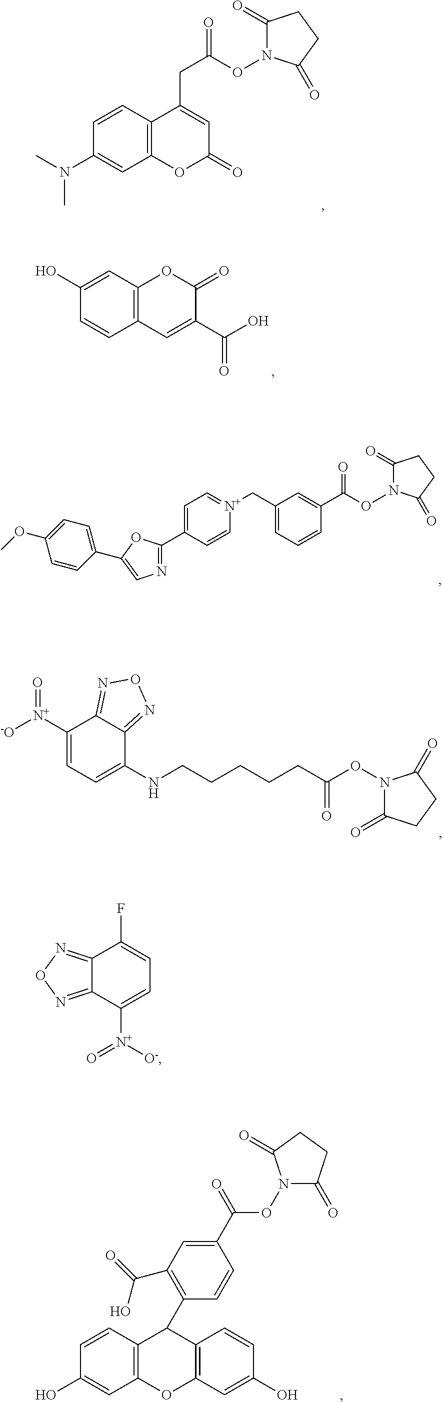

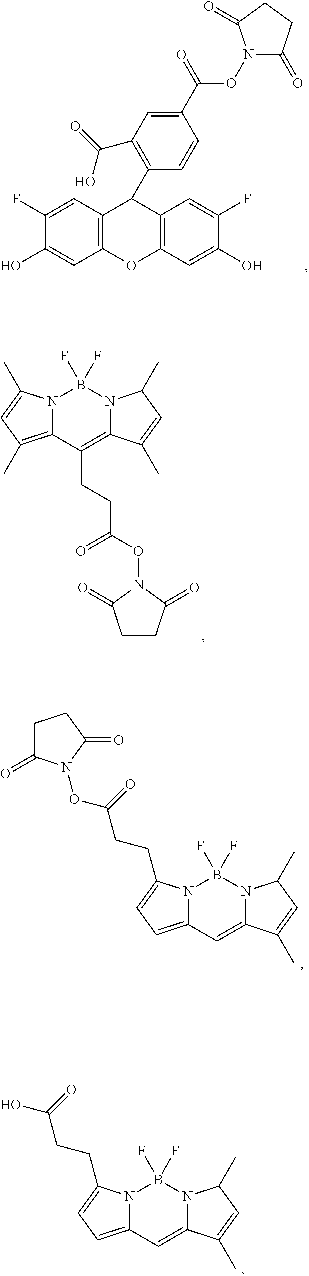

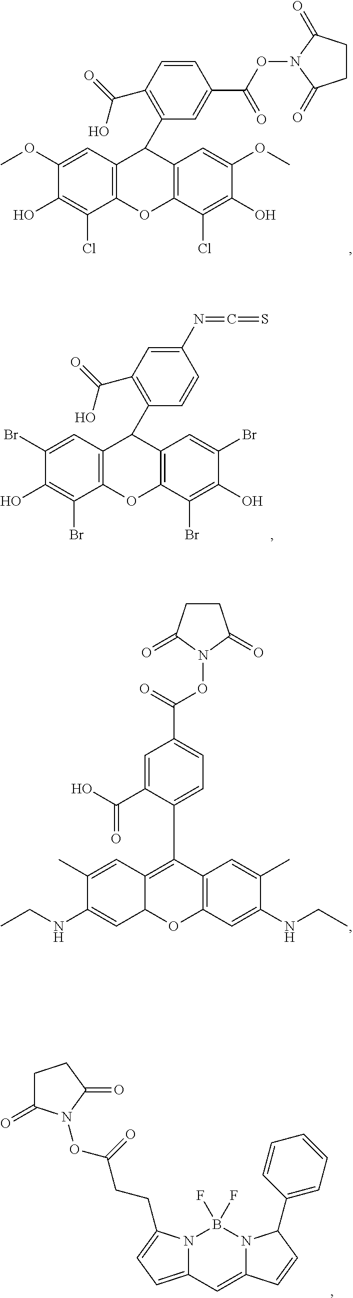

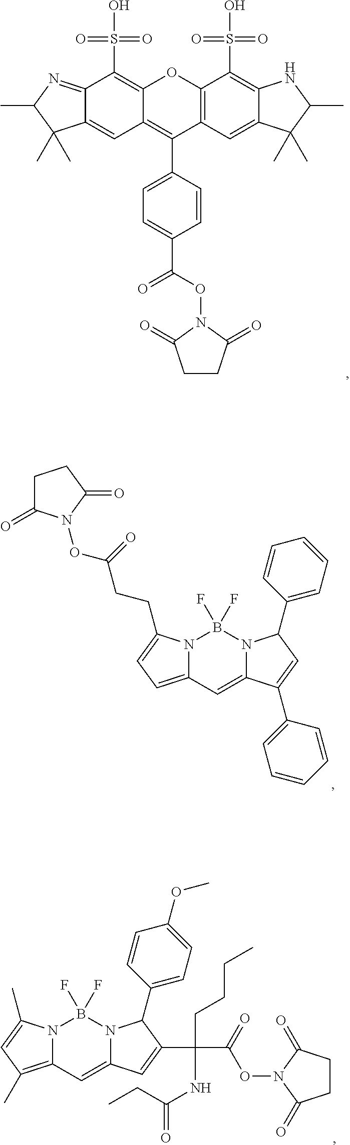

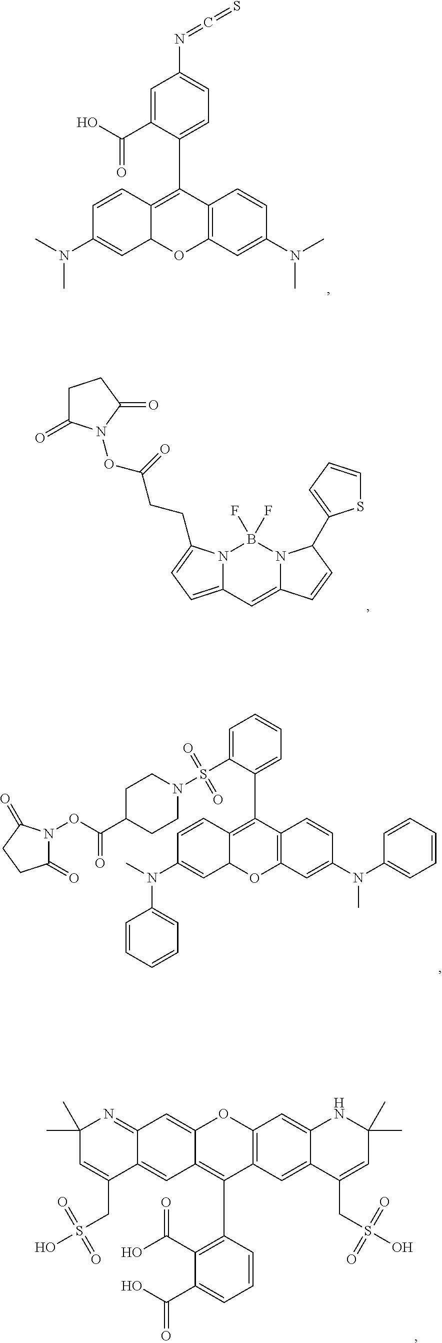

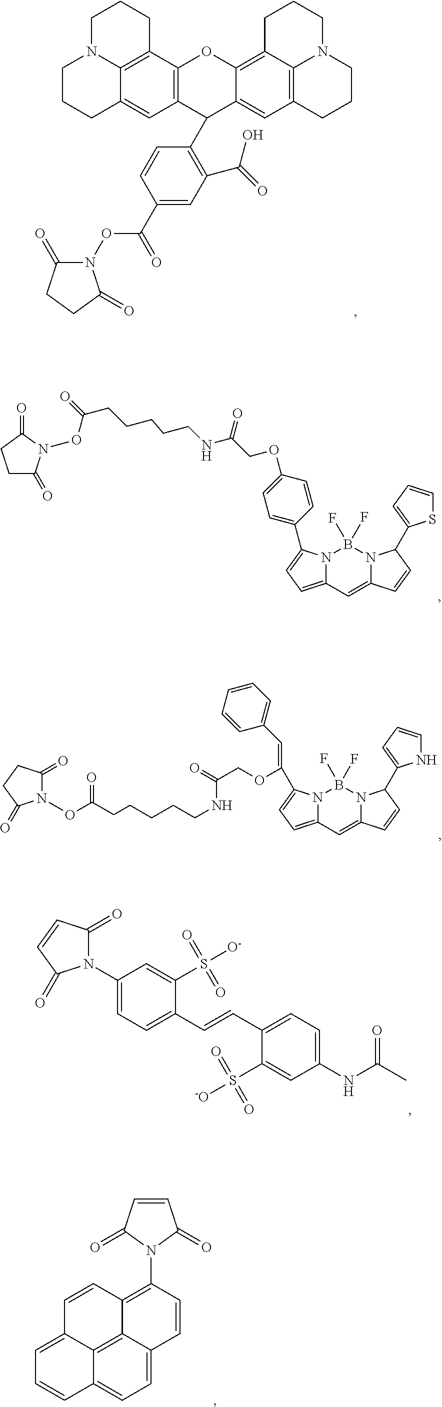

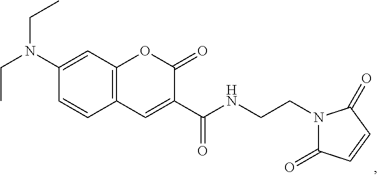 [SMILES: CCN(CC)c1ccc2cc(C(=O)NCCN3C(=O)C=CC3=O)c(=O)oc2c1]

and the like.

Reference probe 4 reacts with the first sample. Analyte 6 combines with the second sample. First calibration probe reacts with the second sample, and the second calibration probe acts with the third sample. Although referred to as first sample, second sample, and third sample, numerical references (e.g., first, second, third) are used merely for convenient reference. As such, each sample (first sample, second sample, third sample) may be identical or different and are generically referred to as “samples.” According to an embodiment, the samples are independently a non-biological sample, biological sample, or a combination thereof that includes an amino acid, protein, peptide, nucleic acid, a polymer bead that is coated with a protein, or a combination thereof. As used herein, a “biological sample” refers to a sample of cell (e.g., eukaryote, prokaryote, and the like), a component of a cell (e.g., organelle, cytoplasm, cell membrane, nuclear membrane, and the like), tissue or fluid isolated from an organism (e.g., animal, human, and the like), such as, for example, blood, plasma, serum, spinal fluid, lymph fluid, the external sections of the skin, respiratory, intestinal, and genitourinary tracts, tears, saliva, milk, cells (e.g., blood cells), tumors, organs, samples of in vitro cell culture constituents, stem cells, liposomes, and the like. Non-limiting exemplary non-biological symbols include organic or inorganic samples, e.g., a polymer, nanoparticle, resin, and the like. In an embodiment, a fourth sample is included, the fourth sample is a blank that includes material such as protein but does not include an analyte probe. The fourth sample is used to subtract a background signal from spectroscopic response (e.g., a fluorescence signal) from other samples, e.g., first sample, second sample, and the like.

According to an embodiment, kit 2 includes the permeation agent. The permeation agent is provided to interact with the samples. In an embodiment, the sample is biological sample and includes a lipid bilayer, and the permeation agent permeates the lipid bilayer so that reference probe 4, analyte probe 6, first calibration probe, or second calibration probe are communicated across the lipid bilayer. Here, the biological sample can be a cell such that the permeation agent permeates the cellular lipid bilayer to communicate the probes from an extracellular environment to an intracellular environment. According to an embodiment, the permeation agent dissolves or emulsifies a portion of the sample (e.g., the lipid bilayer) to communicate the probes into the sample. The permeation agent can form a pore through portion of the sample or form a vesicle that is communicated through a portion of the sample to dispose the probes in the sample.

In an embodiment, the permeation agent includes a detergent, a permeability enhancing peptide, fusion protein, and the like. Detergents include nonionic, anionic, and amphoteric ionic detergent. Nonionic detergents are exemplified by digitonin, Triton X-100, polyoxyethylene alkylether (Brij series), polyoxyethylene sorbitan (Tween series), β-dodecylmaltoside, β-octylglucoside, β-nonylglucoside, β-heptylglucoside, β-octylthioglucoside, sucrose mono-decanoate, sucrose mono-dodecanoate, octyltetraoxyethylene, octylpentaoxyethylene, dodecyloctaoxyethylene, and the like. Anionic detergents are, e.g., taurodeoxycholic acid and the like. Amphoteric ionic detergents are exemplified by N,N-dimethyldecylamine-N-oxide, N,N-dimethyldodecylamine-N-oxide, N,N-dimethyldodecylammonio propanesulfonate, octyl (hydroxyethyl)sulfoxide, octanoyl-N-methylglucamide, nonanoyl-N-methylglucamide, decanoyl-N-methylglucamide, (3-[(3-cholamidepropyl)dimethylammonio]-1-propanesulfonate (CHAPS), and the like. In some embodiments, the detergent is a surfactant as described below. Without wishing to be bound by theory, it is believed that the detergent disrupts membranes by intercalating into phospholipid bilayers and solubilizing lipids and proteins.

The peptides enhance cell permeability, e.g., of a lipid membrane structure. Exemplary peptides include an antimicrobrial peptide, maltose binding protein, chloramphenicol acetyltransferase (CAT), thioredoxin (Trx), and the like. In an embodiment, antimicrobial peptides are oligopeptides or polypeptides that kill microorganisms or inhibit their growth including peptides that result from the cleavage of larger proteins or peptides that are synthesized ribosomally or non-ribosomally. Generally, antimicrobial peptides are cationic molecules with spatially separated hydrophobic and charged regions. Antimicrobial peptides include linear peptides that form an α-helical structure in membranes or peptides that form β-sheet structures optionally stabilized with disulfide bridges in membranes. Representative antimicrobial peptides include, but are not limited to cathelicidins, defensins, dermcidin, and more specifically magainin 2, protegrin, protegrin-1, melittin, LL-37, dermaseptin 01, cecropin, caerin, ovispirin, alamethicin, homologues thereof, variants thereof, and the like. It will be appreciated that antimicrobial peptides include peptides from vertebrates and non-vertebrates, including plants, humans, fungi, microbes, and insects. Antimicrobial peptides include those peptides that increase membrane permeability, for example by forming a pore in the membrane. According to an embodiment, antimicrobial peptides includes (1) those that form a helical structure including an alpha helix and 3,10 helix; (2) those that form a beta structure with disulfide bonds; (3) those that form beta structures without disulfide bonds (i.e., beta strand); (4) those that form both alpha and beta structures; (5) those that are rich in amino-acid residues that include Gly, Trp or Pro; or (6) those produced by vertebrates, non-vertebrates, plants, fungi, or microbes.

The type of the lipid of the lipid membrane structure includes a phospholipid, glycolipid, sterol, long-chain aliphatic alcohol, glycerin fatty acid ester, and the like. It is contemplated that the lipid is a cationic lipid, neutral lipid, or an anionic lipid.

In an embodiment, the kit includes a reducing agent. The reducing agent can increase amounts of reactions that take place between the samples and probes. According to an embodiment, the reducing agent reduces disulfide bonds in the sample to form a thiol group to react with a probe, e.g., reference probe 4, first calibration probe, or second calibration probe. Exemplary reducing agents include 2-mercaptoethylamine-HCl (2-MEA), 2-mercaptoethanol (2-ME), tris(2-carboxyethyl)phosphine (TCEP), cysteine hydrochloride, dithiothreitol (DTT), and the like.

In an embodiment, kit 2 includes a supplemental agent such as a fixative, preservative, cross-linker, solvent, surfactant, and the like. Exemplary fixatives and preservatives include an aldehyde (e.g., formaldehyde, glutaraldehyde, and the like), alcohol (e.g., ethanol, methanol, and the like), and the like that interact with the samples to fix or preserve samples (e.g., a tissue or cell) prior to or at a same time as interaction with reference probe 4, analyte probe 6, first calibration probe, or second calibration probe. In an embodiment, providing the fixative occurs prior to application of kit 2. In some embodiments, providing the fixative occurs after application of kit 2

Without wishing to be bound theory, it is believed that, in some circumstances, the probes permeate into a cell after permeabilization of the cell membrane. In an embodiment, the cell member is permeabilized after fixation such that a cellular content is not denatured or released into a surrounding media, removing a biological context (e.g., position, shape, concentration, and the like) within the cell.

According to an embodiment, kit 2 includes the cross-linker to react with the sample and a probe (reference probe 4, analyte probe 6, first calibration probe, or second calibration probe) in order to couple the sample to the probe. The cross-linker can be carbodiimide that reacts with carboxyl groups of the sample. Exemplary carbodiimides include carbonyldiimidazole (CDI), 1-ethyl-3-(-3-dimethylaminopropyl) carbodiimide hydrochloride (EDC or EDAC), N′,N′-dicyclohexyl carbodiimide (DCC), and the like. In a certain embodiment, the cross-linker reacts with a surface protein of a sample (e.g., a cell) to couple reference probe 4 that includes reactive moiety Q1 having in NHS ester.

According to an embodiment, kit 2 includes a solvent. The solvent can be selected so that components of kit 2 have appreciable solubility in the solvent or is selected to control the available amount of reference probe 4, analyte probe 6, first calibration probe, or second calibration probe in the solvent available to the respective sample. In this regard, the solvent is a polar solvent (e.g., an aqueous solvent) or a nonpolar solvent.

The solvent may include polar protic solvents, polar aprotic solvents, or a combination comprising at least one of these. The solvent may include an electrolyte in the form of a salt, or a pH adjustment agent (e.g., by addition of acid or base), or a buffering agent.

An aqueous solvent is, e.g., water, and organic solvents include an alcohol (e.g., methanol, ethanol, isopropanol, and the like), dimethylsulfone, acetone, an acetate, dimethsulfoxide, dimethylformamide, γ-butyrolactone, tetrahydrofuran, propylene carbonate, ethylene glycol, an ether, an aromatic solvent (e.g., benzene, toluene, p-xylene, ethylbenzene, and the like), or a combination comprising at least one of the foregoing.

Exemplary solvents thus include water including buffered or pH adjusted water; alcohols, such as methanol, ethanol, propanol, isopropanol, butanol, t-butanol, octanol, cyclohexanol, ethylene glycol, ethylene glycol methyl ether, ethylene glycol ethyl ether, ethylene glycol butyl ether, propylene glycol, propylene glycol methyl ether, propylene glycol ethyl ether, diethylene glycol, triethylene glycol, dipropylene glycol, tripropylene glycol, cyclohexanol, and the like; polar aprotic solvents such as dimethylsulfoxide, sulfolane, ethylene carbonate, propylene carbonate, N,N-dimethylformamide, N,N-dimethylacetamide, N-methylpyrrolidone, gamma butyrolactone, and the like; or a combination comprising at least one of the foregoing solvents.

According to an embodiment, a surfactant is included in kit 2 to disperse the probes among a respective sample or in the solvent. Useful surfactants include fatty acids of up to 22 carbon atoms such as stearic acids and esters and polyesters thereof, poly(alkylene glycols) such as poly(ethylene oxide), polypropylene oxide), and block and random poly(ethylene oxide-propylene oxide) copolymers such as those marketed under the trademark PLURONIC by BASF. Other surfactants include polysiloxanes, such as homopolymers and copolymers of poly(dimethylsiloxane), including those having functionalized end groups, and the like. Other useful surfactants include those having a polymeric dispersant having poly(alkylene glycol) side chains, fatty acids, or fluorinated groups such as perfluorinated C₁₋₄ sulfonic acids grafted to the polymer backbone. Polymer backbones include those based on a polyester, a poly(meth)acrylate, a polystyrene, a poly(styrene-(meth)acrylate), a polycarbonate, a polyamide, a polyimide, a polyurethane, a polyvinyl alcohol, or a copolymer comprising at least one of these polymeric backbones. Additionally, the surfactant can be anionic, cationic, zwitterionic, or non-ionic.

Exemplary cationic surfactants include but are not limited to alkyl primary, secondary, and tertiary amines, alkanolamides, quaternary ammonium salts, alkylated imidazolium, and pyridinium salts. Additional examples of the cationic surfactant include primary to tertiary alkylamine salts such as, for example, monostearylammonium chloride, distearylammonium chloride, tristearylammonium chloride; quaternary alkylammonium salts such as, for example, monostearyltrimethylammonium chloride, distearyldimethylammonium chloride, stearyldimethylbenzylammonium chloride, monostearyl-bis(polyethoxy)methylammonium chloride; alkylpyridinium salts such as, for example, N-cetylpyridinium chloride, N-stearylpyridinium chloride; N,N-dialkylmorpholinium salts; fatty acid amide salts such as, for example, polyethylene polyamine; and the like.

Exemplary anionic surfactants include alkyl sulfates, alkyl sulfonates, fatty acids, sulfosuccinates, and phosphates. Examples of an anionic surfactant include anionic surfactants having a carboxyl group such as sodium salt of alkylcarboxylic acid, potassium salt of alkylcarboxylic acid, ammonium salt of alkylcarboxylic acid, sodium salt of alkylbenzenecarboxylic acid, potassium salt of alkylbenzenecarboxylic acid, ammonium salt of alkylbenzenecarboxylic acid, sodium salt of polyoxyalkylene alkyl ether carboxylic acid, potassium salt of polyoxyalkylene alkyl ether carboxylic acid, ammonium salt of polyoxyalkylene alkyl ether carboxylic acid, sodium salt of N-acylsarcosine acid, potassium salt of N-acylsarcosine acid, ammonium salt of N-acylsarcosine acid, sodium salt of N-acylglutamic acid, potassium salt of N-acylglutamic acid, ammonium salt of N-acylglutamic acid; anionic surfactants having a sulfonic acid group; anionic surfactants having a phosphonic acid; and the like.

The nonionic surfactant can be, e.g., ethoxylated fatty alcohols, alkyl phenol polyethoxylates, fatty acid esters, glycerol esters, glycol esters, polyethers, alkyl polyglycosides, amineoxides, or a combination thereof. Exemplary nonionic surfactants include fatty alcohols (e.g., cetyl alcohol, stearyl alcohol, cetostearyl alcohol, oleyl alcohol, and the like); polyoxyethylene glycol alkyl ethers (e.g., octaethylene glycol monododecyl ether, pentaethylene glycol monododecyl ether, and the like); polyoxypropylene glycol alkyl ethers (e.g., butapropylene glycol monononyl ether); glucoside alkyl ethers (e.g., decyl glucoside, lauryl glucoside, octyl glucoside); polyoxyethylene glycol octylphenol ethers (e.g., Triton X-100 (octyl phenol ethoxylate)); polyoxyethylene glycol alkylphenol ethers (e.g., nonoxynol-9); glycerol alkyl esters (e.g., glyceryl laurate); polyoxyethylene glycol sorbitan alkyl esters (e.g., polysorbates such as sorbitan monolaurate, sorbitan monopalmitate, sorbitan monostearate, sorbitan tristearate, sorbitan monooleate, and the like); sorbitan alkyl esters (e.g., polyoxyethylene sorbitan monolaurate, polyoxyethylene sorbitan monopalmitate, polyoxyethylene sorbitan monostearate, polyoxyethylene sorbitan monooleate, and the like); cocamide ethanolamines (e.g., cocamide monoethanolamine, cocamide diethanolamine, and the like); amine oxides (e.g., dodecyldimethylamine oxide, tetradecyldimethylamine oxide, hexadecyl dimethylamine oxide, octadecylamine oxide, and the like); block copolymers of polyethylene glycol and polypropylene glycol (e.g., poloxamers available under the trade name Pluronics, available from BASF); polyethoxylated amines (e.g., polyethoxylated tallow amine); polyoxyethylene alkyl ethers such as polyoxyethylene stearyl ether; polyoxyethylene alkylene ethers such as polyoxyethylene oleyl ether; polyoxyalkylene alkylphenyl ethers such as polyoxyethylene nonylphenyl ether; polyoxyalkylene glycols such as polyoxypropylene polyoxyethylene glycol; polyoxyethylene monoalkylates such as polyoxyethylene monostearate; bispolyoxyethylene alkylamines such as bispolyoxyethylene stearylamine; bispolyoxyethylene alkylamides such as bispolyoxyethylene stearylamide; alkylamine oxides such as N,N-dimethylalkylamine oxide; and the like

Zwitterionic surfactants (which include a cationic and anionic functional group on the same molecule) include, for example, betaines, such as alkyl ammonium carboxylates (e.g., [(CH₃)₃N⁺—CH(R)COO⁻] or sulfonates (sulfo-betaines) such as [RN⁺(CH₃)₂(CH₂)₃SO₃₋], where R is an alkyl group). Examples include n-dodecyl-N-benzyl-N-methylglycine [C₁₂H₂₅N⁺(CH₂C₆H₅)(CH₃)CH₂COO⁻], N-allyl N-benzyl N-methyltaurines [CₙH₂₊₁N⁺(CH₂C₆H₅)(CH₃)CH₂CH₂SO₃⁻].

Certain amount of various components are used as probes and other materials. A fixation buffer includes, e.g., 3.7% to 4% paraformaldehyde, based on a total volume of the buffer. The analyte probe includes, e.g., from 1 microgram per milliliter (μg/ml) to 100 μg/m and specifically 20 μg/m in a buffer (e.g., PBS). The permeabilization agent includes e.g., Triton-X 100 diluted to 0.1% in a buffer (e.g., PBS), based on a total volume of the solution.

In an embodiment, the reference probe is 3,6-diamino-9-[2-carboxy-4-[[[5-(2,5-dihydro-2,5-dioxo-1H-pyrrol-1-yl)pentyl]amino]carbonyl]phenyl]-4,5-disulfo-Xanthylium (commercially available under trade name Alexa Fluor 488 Maleimide from Life Technologies). Additionally, the first calibration probe or second calibration probe independently can include a structure such as:

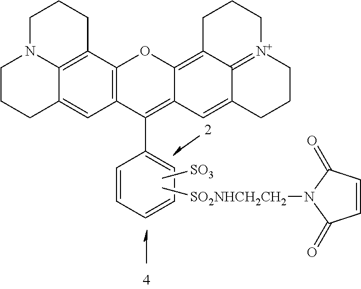

(commercially available under trade name Texas Red Maleimide from Life Technologies) or

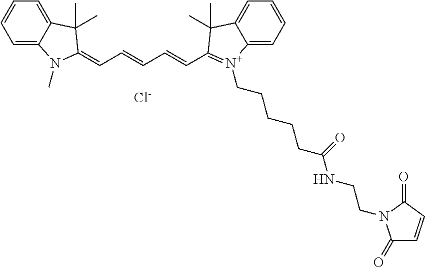 [SMILES: CN1/C(=C/C=C/C=C/C2=[N+](CCCCCC(=O)NCCN3C(=O)C=CC3=O)c3ccccc3C2(C)C)C(C)(C)c2ccccc21 ; [Cl-]]

(commercially available under trade name Cy5 Maleimide from Lumiprobe) (e.g., the first or second calibration probe). A concentration of these probes can be from 0.1 micromolar (μM) to 500 μM. In an embodiment, a number of different concentration of these probes is selected over this concentration range to determine a maximum saturating fluorescence intensity or a concentration range that produces a fluorescence intensity that produces a fluorescence intensity that is least sensitive to fluctuations in fluorophore concentration used in labeling. It is contemplated that such staining (e.g., for mammalian cells) occurs for a concentration from 20 μM to 200 μM. In an embodiment, the fluorescent maleimide (such as a probe recited above) is diluted with 95% to 99% unlabeled maleimide, e.g., N-ethyl maleimide to provide a total maleimide (unlabeled plus labeled) concentration that is within a selected concentration range. Without wishing to be bound by theory, it is believed that adding an unlabeled maleimide reduces self-quenching of the fluorophores. Reducing self-quenching provides for maintaining a direct relationship between fluorescence binding and actual protein amount. Adding unlabeled maleimide can reduce a fluorescence intensity of a labeled sample, which provides an intensity that is typical for flow cytometry measurement.

According to an embodiment, a probe (e.g., reference probe, analyte probe, marker, calibration probe, or a combination thereof) are used to stain a sample, e.g., the first sample, second sample, third sample, or a combination thereof. In an embodiment, a process for staining includes forming a reference composition by reacting a reference probe and a first sample to form a reference and forming an analyte composition by combining an analyte probe and a second sample to form an analyte. The process further includes introducing a first calibration probe into the analyte composition and forming a first calibrant by reacting the first calibration probe and the second sample. The process also includes forming a calibration composition by reacting a second calibration probe and a third sample to form a second calibrant.

According to an embodiment, the probes stain samples such as cells. Once stained using the probes to produce the reference, analyte, first calibrant, or second calibrant, the samples are subjected to optical analysis such as by excitation with a wavelength in detection of absorbance, fluorescence, and the like. In some embodiments, the cells in the samples are observed under a microscope, the presence of the reference, analyte, first calibrant, or second calibrant being diagnostic for a presence, e.g., of protein in the samples. Another use of the probes is in immunoassays or competitive protein binding assays, where the probes serve as fluorescent labels.

In a particular embodiment, the reference and analyte have a substantially similar absorption and fluorescence spectra due to inclusion of the same spectrally active group A1 in the reference and analyte. Likewise, in a certain embodiment, the first calibrant and second calibrant have a substantially similar absorption and fluorescence spectra due to inclusion of the same spectrally active group A2 in the first calibration probe and second calibration probe. Such details regarding the spectrally active groups A1 and A2 respectively in the reference, analyte, first calibrant, and second calibrant provide normalization of optical data, e.g., from fluorescence spectra of the analyte with respect to optical data, e.g., fluorescence spectra, of the reference, first calibrant, and second calibrant.

In an embodiment, a process for obtaining a normalized signal from an analyte includes combining a reference probe and a first sample to produce a reference composition, reacting the reference probe and the first sample to form a reference in the reference composition, determining a reference signal from the reference in response to subjecting the reference to radiation, combining an analyte probe and a second sample to produce an analyte composition, contacting the analyte probe and the second sample to form an analyte in the second composition, determining an analyte signal from the analyte in response to subjecting the analyte to radiation, and obtaining the normalized signal by normalizing the analyte signal to the reference signal. The process further includes introducing a first calibration probe in the analyte composition and reacting the first calibration probe and the second sample to form a first calibrant in the analyte composition. A first calibration signal is determined from the first calibrant in response to subjecting the first calibrant to radiation. Additionally, the process includes combining a second calibration probe and a third sample to produce a calibration composition, reacting the second calibration probe and the third sample to form a second calibrant in the calibration composition, and determining a second calibration signal from the second calibrant in response to subjecting the second calibrant to radiation. In some embodiments, obtaining the normalized signal includes calibrating the analyte signal to the first calibration signal and the second calibration signal. In certain embodiments, forming the analyte probe occurs by reacting a marker and a reference probe.

As used herein, “combining” refers to attachment or association of the analyte probe and second sample, whether specific or non-specific, as a result of chemical reaction or as a result of direct or indirect physical interactions, van der Waals interactions, London forces, or weak interactions, or as a result of magnetic, electrostatic, or electromagnetic interaction such as a binding event between the analyte probe and the second sample. As used herein, the term “binding event” refers to an interaction or association between a plurality of molecular structures such as the analyte and the second sample. The interaction can occur when the two molecular structures are in direct or indirect physical contact or when the two structures are physically separated but electromagnetically interacting. Examples of binding events include ligand/receptor, antigen/antibody, enzyme/substrate, DNA/DNA, DNA/RNA, RNA/RNA, hybrids, nucleic acid mismatch, complementary nucleic acids and nucleic acid/proteins. The binding event can be specific or non-specific, and bonds formed include covalent bonds, hydrogen bonds, immunological binding, Van der Waals forces, ionic forces, or other types of binding.

Detection of the analyte, reference, first calibrant, or second calibrant includes detection by fluorescence, chemiluminescence, bioluminescence, colorimetry, absorbance, or quantum dot methods. Detection is due to, e.g., a response (such as an optical response) from spectrally active groups A1 and A2 incorporated into the analyte, reference, first calibrant, and second calibrant respectively from the analyte probe, reference probe, first calibration probe, and the second calibration probe. The response is due to subjecting the analyte, reference, first calibrant, or second calibrant to a wavelength of excitation, e.g., an ultraviolet wavelength, visible wavelength, near infrared wavelength, infrared wavelength, microwave wavelength, and the like. In certain embodiments, spectrally active group A1 or A2 is selected to correspond to a wavelength of excitation or detection. According to an embodiment, in addition to spectrally active groups A1 and A2 disclosed above, spectrally active group A1 or A2 can optionally or alternatively include radioisotopes, fluorophores, chemiluminescers, chemiluminophores, bioluminescers, enzymes, antibodies, and particles such as magnetic particles and quantum dots. A variety of schemes for detection of such material is described in M. Schena and R. W. Davis, DNA Microarrays: A Practical Approach (M. Schena ed., Oxford University Press 1999), which is incorporated herein by reference in its entirety.

For fluorescence detection, excitation light may be provided by a light source (e.g., a light emitting diode, laser, flash lamp, and the like) to the composition (e.g., reference composition, analyte composition, or calibration composition), and a detector (e.g., a photodetector such as a CMOS sensor, a charge coupled device, photomultiplier, photodiode, and the like) can detect the fluorescence emitted by the composition. An optical filter can be disposed between the light source and the composition, between the composition in the detector, or a combination thereof. In certain embodiments, the compositions can be disposed in a plurality of wells of an array. Here, optical filters can be disposed between the array spots and photodiodes to remove the excitation wavelength and to select for detection the emitted fluorescence from the compositions in the wells of the array. Optical filters can include low-pass, band-pass, high-pass, and “mirror” elements that pass certain wavelengths and reflect others.

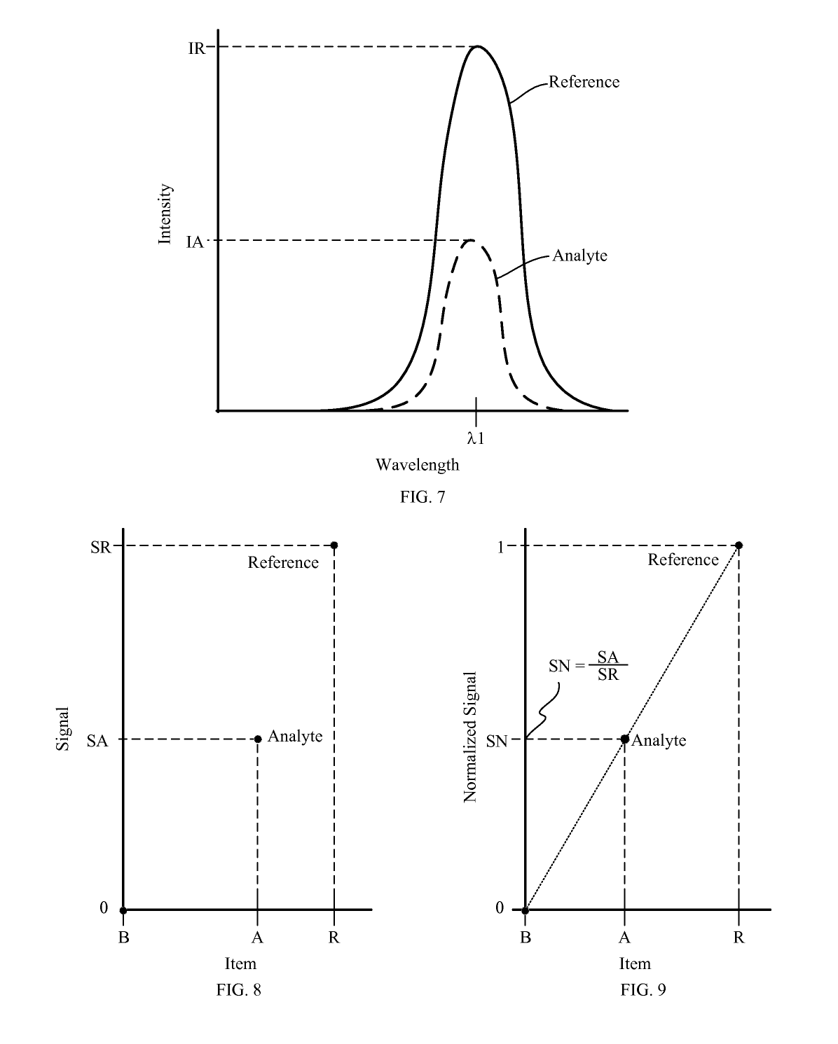

With reference to FIGS. 6, 7, 8, and 9, in an embodiment, a process for obtaining a normalized analyte signal includes reacting a reference probe and first sample (step 100) to form the reference (step 102), subjecting the reference to radiation (step 104), and detecting an intensity of fluorescence (IR) from the reference. FIG. 7 shows an exemplary fluorescence emission spectrum for the reference having a peak intensity at first wavelength λ1 due to a presence of spectrally active group A1 in the reference. The process also includes combining the analyte probe and the second sample (step 108), forming the analyte (step 110), subjecting the analyte to radiation (step 112), and detecting an intensity of fluorescence IA from the analyte, which is also shown in FIG. 7 wherein the analyte has a peak intensity at first wavelength λ1 due to the presence of spectrally active group A1 in the analyte. Additionally, the process optionally can include subjecting a blank (unlabeled) sample B to radiation and detecting an intensity of fluorescence (IB) from the blank sample B. In this manner, background fluorescence collected from blank sample B can be subtracted from IA and IR. The process further includes determining reference signal SR from IR (step 106), e.g., by integrating IR (potentially optical or electronic filtered) over a region of wavelength and exposure time (e.g., of a camera) or residence time in a beam (e.g. flow cytometry) to obtain total fluorescence due to the reference, corrected against the background fluorescence from blank sample B. Moreover, the process includes determining analyte signal SA from IA (step 114), e.g., by integrating IA (potentially optical or electronic filtered) over a region of wavelength and exposure time (e.g. of a camera) or residence time in a beam (e.g. flow cytometry) to obtain total fluorescence due to the analyte, corrected against the background fluorescence from blank sample B. FIG. 8 shows a graph of signal versus component (i.e., blank B, analyte A, and reference R) with reference signal SR from the reference and analyte signal SA from the analyte. It should be appreciated that since intensities IA, IR, and IB are corrected against the background fluorescence from simple blank B, the signal from sample blank B is shown as having a zero value in FIGS. 8 and 9. Normalized analyte signal SN is obtained by dividing analyte signal SA by reference signal SR, i.e.: SN=SA/SR as shown in FIG. 9.

In an embodiment, normalizing an analyte signal includes acquiring a fluorescence spectrum for a reference (reference spectrum), acquiring a fluorescence spectrum for an analyte (analyte spectrum), optionally acquiring a fluorescence spectrum for a blank (background spectrum), optionally correcting the reference spectrum and analyte spectrum with respect to the background spectrum, integrating the intensity over a region of wavelength and exposure time or residence time of the reference spectrum to obtain a reference signal (i.e., integrated fluorescence from the reference), integrating the intensity over a region of wavelength and exposure time or residence time of the analyte spectrum to obtain an analyte signal (i.e., integrated fluorescence from the analyte), optionally integrating the intensity over a region of wavelength and exposure time or residence time of the background spectrum to obtain the background signal (i.e., integrated fluorescence from the blank), optionally correcting the reference signal and the analyte signal with respect to the background signal (e.g., by subtracting the background signal from the reference signal and the analyte signal), and normalizing the analyte signal with respect to the reference signal (e.g., by dividing the analyte signal by the reference signal) to obtain the normalized analyte signal SN.

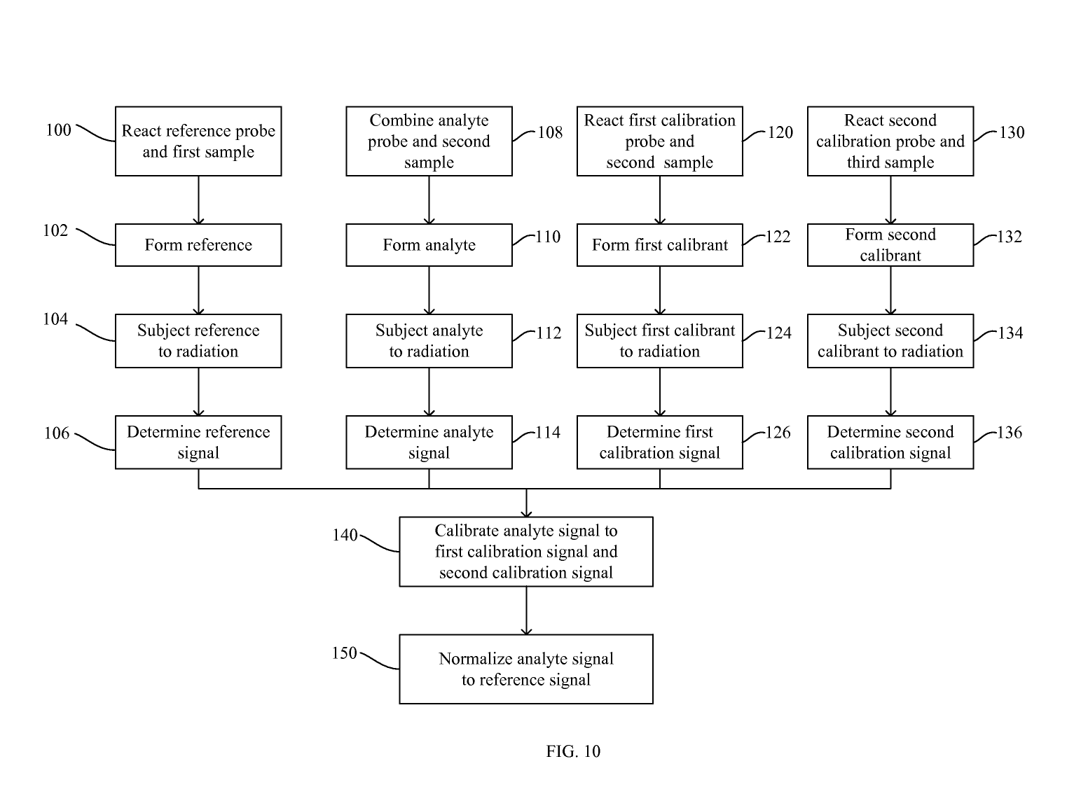

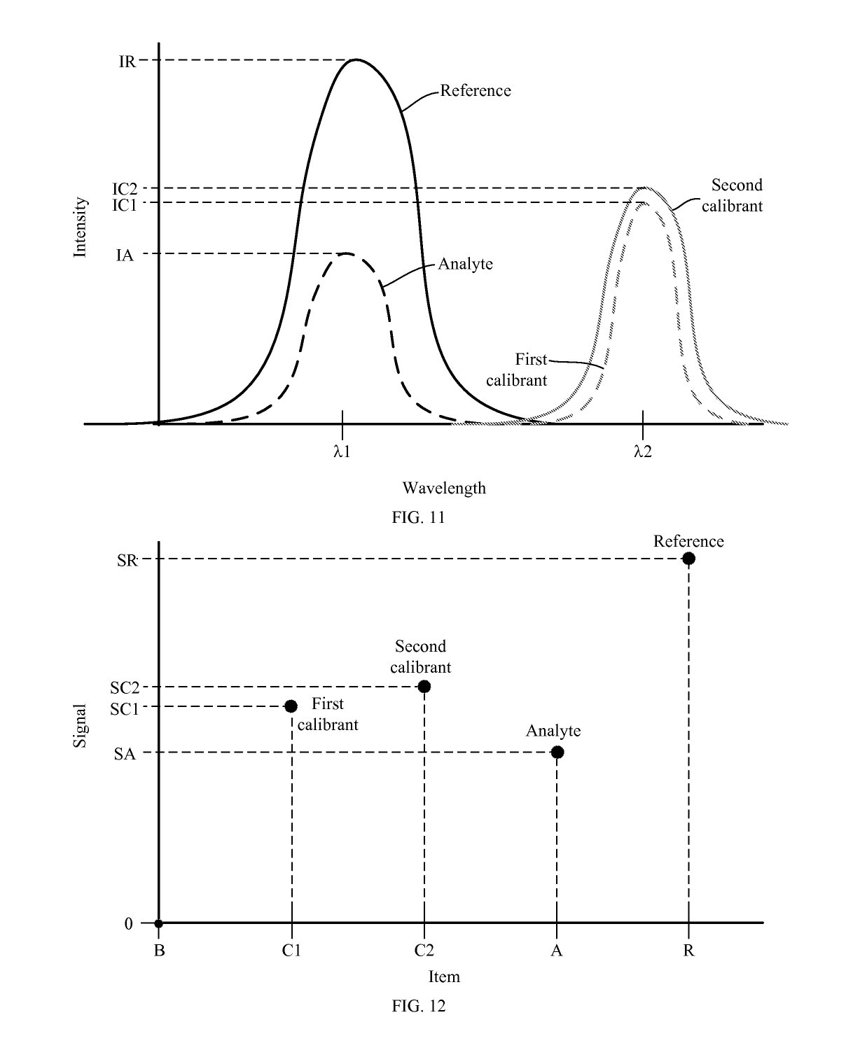

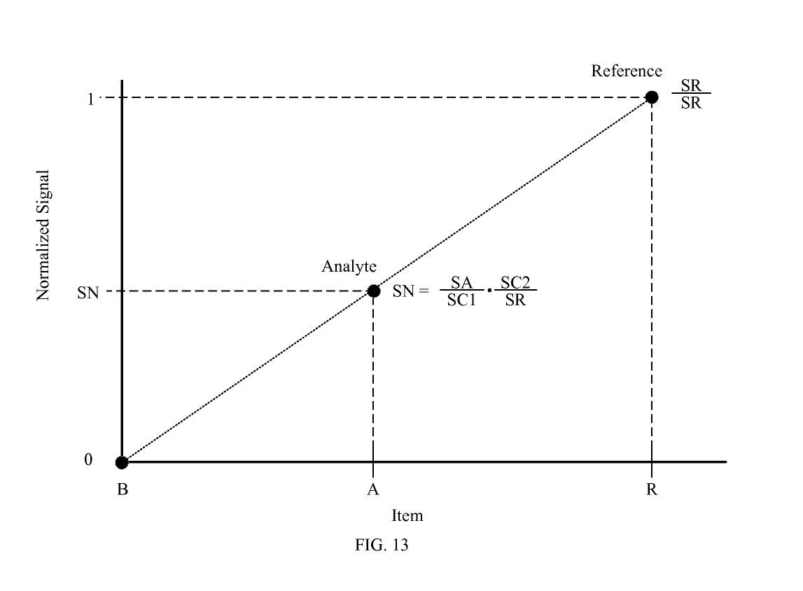

With reference to FIGS. 10, 11, 12, and 13, according to an embodiment, a process for obtaining a normalized analyte signal includes reacting a reference probe and first sample (step 100) to form the reference (step 102), subjecting the reference to radiation (step 104), and detecting an intensity of fluorescence (IR) from the reference. FIG. 11 shows an exemplary fluorescence spectrum for the reference having a peak intensity at first wavelength λ1 due to a presence of spectrally active group A1 in the reference. The process also includes combining the analyte probe and the second sample (step 108), forming the analyte (step 110), subjecting the analyte to radiation (step 112), and detecting an intensity of fluorescence IA from the analyte, which is also shown in FIG. 11, wherein the analyte has a peak intensity at first wavelength λ1 due to the presence of spectrally active group A1 in the analyte.

The process further includes reacting the first calibration probe and the second sample (step 120) to form the first calibrant (step 122), subjecting the first calibrant to radiation (step 124), and detecting an intensity of fluorescence (IC1) from the first calibrant. FIG. 11 shows an exemplary fluorescence spectrum for the first calibrant having a peak intensity at second wavelength λ2 due to a presence of spectrally active group A2 in the first calibrant. The process also includes reacting a second calibration probe with a third sample (step 130) to form the second calibrant (step 132), subjecting the second calibrant to radiation (step 134), and detecting an intensity of fluorescence (IC2) from the second calibrant. FIG. 11 shows an exemplary fluorescence spectrum for the second calibrant having a peak intensity at second wavelength λ2 due to a presence of spectrally active group A2 in the first calibrant.

Additionally, the process optionally can include subjecting a blank sample B to radiation and detecting an intensity of fluorescence (IB) from the blank sample B. In this manner, background fluorescence collected from blank sample B be can be subtracted from IA, IR, IC1, and IC2. The process further includes determining reference signal SR from IR (step 106), e.g., by integrating IR (potentially optical or electronic filtered) over a region of wavelength and exposure time (e.g. of a camera) or residence time in a beam (e.g. flow cytometry) to obtain total fluorescence due to the reference, corrected against the background fluorescence from blank sample B. Moreover, the process includes determining analyte signal SA from IA (step 114), e.g., by integrating IA (potentially optical or electronic filtered) over a region of wavelength and exposure time (e.g. of a camera) or residence time in a beam (e.g. flow cytometry) to obtain total fluorescence due to the analyte, corrected against the background fluorescence from blank sample B. The process also includes determining first calibration signal SC1 from IC1 (step 126), e.g., by integrating IC1 (potentially optical or electronic filtered) over a region of wavelength and exposure time (e.g. of a camera) or residence time in a beam (e.g. flow cytometry) to obtain total fluorescence due to the first calibrant, corrected against the background fluorescence from blank sample B. Moreover, the process includes determining a second calibration signal SC2 from IC2 (step 136), e.g., by integrating IC2 (potentially optical or electronic filtered) over a region of wavelength and exposure time (e.g. of a camera) or residence time in a beam (e.g. flow cytometry) to obtain total fluorescence due to the second calibrant, corrected against the background fluorescence from blank sample B.

FIG. 12 shows a graph of signal versus component (i.e., blank B, analyte A, reference R, first calibrant C1, and second calibrant C2) with reference signal SR from the reference, analyte signal SA from the analyte, first calibration signal SC1 from the first calibrant, and second calibration signal SC2 from the second calibrant. It should be appreciated that since intensities IA, IR, IB, IC1, and IC2 are corrected against the background fluorescence from simple blank B, the signal from sample blank B is shown as having a zero value in FIGS. 12 and 13. Normalized analyte signal SN (see FIG. 13) is obtained by dividing analyte signal SA by reference signal SR and the quotient (i.e. SA/SR) multiplying by a ratio of first calibration signal SC1 to second calibration signal SC2 (i.e., SC1/SC2). Thus, normalized analyte signal SN is determined as in formula 10.

$$SN = \frac{SA}{SC 1} \cdot \frac{SC 2}{SR} \qquad ( 10 )$$

In an embodiment, normalizing an analyte signal includes acquiring a fluorescence spectrum for a reference (reference spectrum); acquiring a fluorescence spectrum for an analyte (analyte spectrum); acquiring a fluorescence spectrum for a first calibrant (first calibration spectrum); acquiring a fluorescence spectrum for a second calibrant (second calibration spectrum); optionally acquiring a fluorescence spectrum for a blank (background spectrum); optionally correcting the reference spectrum, analyte spectrum, first calibration spectrum, and second calibration spectrum with respect to the background spectrum; integrating the intensity over a region of wavelength and exposure time or residence time of the reference spectrum to obtain a reference signal (i.e., integrated fluorescence from the reference); integrating the intensity over a region of wavelength and exposure time or residence time of the analyte spectrum to obtain an analyte signal (i.e., integrated fluorescence from the analyte); integrating the intensity over a region of wavelength and exposure time or residence time of the first calibration spectrum to obtain a first calibration signal (i.e., integrated fluorescence from the first calibrant); integrating the intensity over a region of wavelength and exposure time or residence time of the second calibration spectrum to obtain a second calibration signal (i.e., integrated fluorescence from the second calibrant); optionally integrating the intensity over a region of wavelength and exposure time or residence of the background spectrum to obtain the background signal (i.e., integrated fluorescence from the blank); optionally correcting the reference signal, analyte signal, first calibration signal, and second calibration signal with respect to the background signal (e.g., by subtracting the background signal from the reference signal, analyte signal, first calibration signal, and second calibration signal); and normalizing the analyte signal with respect to the reference signal, first calibration signal, and second calibration signal (e.g., by dividing the analyte signal by the reference signal and multiplying the resulting quotient by a ratio of the second calibration signal to the first calibration signal) to obtain the normalized analyte signal SN.

The probes, kit, and processes herein have advantageous benefits and uses. The reference and analyte provide comparability of biomarker measurements between cell samples and across laboratories. A measurement of total cellular protein is obtained using the reference with or without the calibrants. The reference and analyte are advantageously applied to microscopy and flow cytometry. Beneficially, a process for staining and obtaining the normalized signal therefrom is near universal and instrument independent. As such, articles and processes herein provide a comparison of different therapeutic products and facilitation of design and evaluation of preclinical trials. Furthermore, the analyte in reference to provide for staining and obtaining a normalized signal that is substantially insensitive to concentrations and staining times.

According to an embodiment, the probes are used in combination with flow cytometry. Flow cytometry can be used fir detecting particles (e.g., samples, probes, calibrants, reference, analyte, and the like) suspended in a stream of a fluid. Flow cytometry provides simultaneous multiparametric analysis of physical or chemical characteristics of a cell flowing through an optical/electronic detection member of the flow cytometer. A beam of light, e.g., laser light, at a wavelength is directed onto a hydrodynamically focused stream of the fluid, A plurality of detectors is directed at a position where the stream passes through the light. A detector can be arranged in-line with the light beam (as in forward scatter mode or FSC), and a plurality of detectors can be arranged perpendicular to the light beam (as in side scatter mode or SSC). A fluorescent detector can be included in the flow cytometer.

Each suspended particle (e.g., the reference, analyte, first calibrant, second calibrant) passing through the light beam scatters light, and spectrally active groups (e.g., A1 or A2) in the particle may be excited and emit light at a longer wavelengths than the light beam. The scattered and fluorescent light is detected by the detectors. By analyzing fluctuations in brightness at each detector (one for each fluorescent emission peak), physical structure and chemical composition of particles are determined. In an embodiment, FSC signals correlate with the cell size, and SSC signals depend on an inner property of a particle, such as shape of the nucleus, the amount and type of cytoplasmic granules, or membrane roughness. Here, fluorescence detection can be used to produce normalized analyte signals.

In an embodiment, the flow cytometer analyzes several thousand particles every second in real time and, if the cytometer also has sorting functions, separates and isolates particles with selected properties. According to an embodiment, obtaining the normalized analyte signal coupled to flow cytometry provides high-throughput automated quantification or separation of parameters for a plurality of cells during each analysis session. Flow cytometers can have multiple lasers or fluorescence detectors so that a plurality of spectrally active groups can be simultaneously applied to samples and selectively monitored to determine sample properties. In a certain embodiment, a flow cytometer obtaining the normalized analyte signal provides detection of samples with a plurality of spectrally active groups from the reference probe, analyte probe, first calibration probe, or second calibration probe. In some embodiments, the flow cytometer sorts or isolates different samples, such as by size, different markers, surface proteins, intracellular proteins, and the like.

Various terms are used herein. As used herein, “alkenyl” means a linear or branched chain, monovalent hydrocarbon group having at least one carbon-carbon double bond (e.g., ethenyl (—HC═CH₂)).

As used herein, “alkenylene” means a linear or branched chain, divalent hydrocarbon group having at least one carbon-carbon double bond (e.g., ethenylene (—HC═CH—)), optionally substituted with one or more substituents where indicated, provided that the valence of the alkyl group is not exceeded.

As used herein, “alkoxy” means an alkyl group that is linked via an oxygen (i.e., —O-alkyl). Nonlimiting examples of C1 to C30 alkoxy groups include methoxy groups, ethoxy groups, propoxy groups, isobutyloxy groups, sec-butyloxy groups, pentyloxy groups, iso-amyloxy groups, and hexyloxy groups.

As used herein, “alkyl” means a linear or branched chain, saturated, monovalent hydrocarbon group (e.g., methyl or hexyl), optionally substituted with one or more substituents where indicated, provided that the valence of the alkyl group is not exceeded. Alkyl groups include, for example, groups having from 1 to 50 carbon atoms (C1 to C50 alkyl).

As used herein, “alkylamine” means a group of the formula -Q-N(Rw)(Rz), wherein Q is a C1 to C15 alkenylene, and Rw and Rz are independently hydrogen, a C1 to C14 alkyl, a C1 to C14 alkenyl, a C1 to C14 alkynyl, a C3 to C14 cycloalkyl or a C6 to C14 aryl; such that the total number of carbon atoms in Q, Rw, and Rz is from 1 to 15.

As used herein, “akylaryl” means an alkyl group covalently linked to a substituted or unsubstituted aryl group that is linked to a compound.

As used herein, “alkylene” means a linear or branched chain, saturated, divalent aliphatic hydrocarbon group, (e.g., methylene (—CH₂—) or, propylene (—(CH₂)₃—)).

As used herein, “alkylene” means a linear, branched or cyclic divalent aliphatic hydrocarbon group, and may have from 1 to about 18 carbon atoms, more specifically 2 to about 12 carbons. Exemplary alkylene groups include methylene (—CH₂—), ethylene (—CH₂CH₂—), propylene (—(CH₂)₃—), cyclohexylene (—C₆H₁₀—), methylenedioxy (—O—CH₂—O—), or ethylenedioxy (—O—(CH₂)₂—O—).

As used herein, “alkynyl” means a linear or branched chain, monovalent hydrocarbon group having at least one carbon-carbon triple bond (e.g., ethynyl).

As used herein, “alkynylene” means a linear or branched chain divalent aliphatic hydrocarbon that has one or more unsaturated carbon-carbon bonds, at least one of which is a triple bond (e.g., ethynylene).

As used herein, “amide” means a group of the formula —C(O)—N(Rx)(Ry) or —N—C(O)—Rx, wherein Rx is an alkyl, an alkenyl, an alkynyl, a cycloalkyl or an aryl group; and Ry is hydrogen or any of the groups listed for Rx.

As used herein, “amine” refers to the general formula NR′R′, wherein each R′ is independently hydrogen, an alkyl group, or an aryl group.

As used herein, “aryl” refers to a hydrocarbon group having an aromatic ring, and includes monocyclic and polycyclic hydrocarbons wherein the additional ring(s) of the polycyclic hydrocarbon may be aromatic or nonaromatic (e.g., phenyl or napthyl).

As used herein, “arylalkyl” means a substituted or unsubstituted aryl group covalently linked to an alkyl group that is linked to a compound (e.g., a benzyl is a C7 arylalkyl group).

As used herein, “arylalkylene” group is an aryl group linked via an alkylene moiety. The specified number of carbon atoms (e.g., C7 to C30) means the total number of carbon atoms present in both the aryl and the alkylene moieties. Representative arylalkyl groups include, for example, benzyl groups.

As used herein, “arylene” means a divalent group formed by the removal of two hydrogen atoms from one or more rings of an arene, wherein the hydrogen atoms may be removed from the same or different rings (e.g., phenylene or napthylene).

As used herein, “aryloxy” means an aryl moiety that is linked via an oxygen (i.e., —O-aryl).

As used herein, an asterisk (i.e., “*”) denotes a point of attachment, e.g., a position linked to the same or different atom or chemical formula.

As used herein, “cycloalkylene” means a divalent radical formed by the removal of two hydrogen atoms from one or more rings of a cycloalkyl group (a nonaromatic hydrocarbon that comprises at least one ring).

As used herein, “cycloalkynyl” means an aliphatic monocyclic or polycyclic group having at least one carbon-carbon triple bond, wherein all ring members are carbon (e.g., cyclohexynyl).

As used herein, “cycloalkenylene” means an aliphatic 5-15-membered monocyclic or polycyclic, divalent radical having at least one carbon-carbon double bond, which comprises one or more rings connected or bridged together. Unless disclosed otherwise, the cycloalkenylene radical can be linked at any desired carbon atom provided that a stable structure is obtained. If the cycloalkenylene radical is substituted, this may be so at any desired carbon atom, once again provided that a stable structure is obtained. Examples thereof are cyclopentenylene, cyclohexenylene, cycloheptenylene, cyclooctenylene, cyclononenylene, cyclodecenylene, norbornenylene, 2-methylcyclopentenylene, 2-methylcyclooctenylene.

As used herein, “cycloalkyl” means a monovalent group having one or more saturated and/or partially saturated rings in which all ring members are carbon (e.g., cyclopropyl, cyclobutyl, cyclopentyl, cyclohexyl, cycloheptyl, cyclooctyl, and partially saturated variants of the foregoing, such as cycloalkenyl groups (e.g., cyclohexenyl) or cycloalkynyl groups.

As used herein, “cycloalkenyl” means a monovalent group having one or more rings and one or more carbon-carbon double bond in the ring, wherein all ring members are carbon (e.g., cyclopentyl and cyclohexyl).

As used herein, “halogen” means one of the elements of group 17 of the periodic table (e.g., fluorine, chlorine, bromine, iodine, and astatine).

As used herein, the prefix “hetero” means that the compound or group includes an atom that is a heteroatom (e.g., 1, 2, or 3 heteroatom(s)), wherein the heteroatom(s) is each independently N, O, S, Si, or P.

Reference herein is made to various heterocyclic groups. Within such groups, the term “hetero” means a group that comprises at least one ring member that is a heteroatom (e.g., 1 to 4 heteroatoms, each independently N, O, S, P, or Si). In each instance, the total number of ring members may be indicated (e.g., a 3- to 10-membered heterocycloalkyl). If multiple rings are present, each ring is independently aromatic, saturated, or partially unsaturated, and multiple rings, if present, may be fused, pendant, spirocyclic or a combination thereof. Heterocycloalkyl groups comprise at least one non-aromatic ring that contains a heteroatom ring member. Heteroaryl groups comprise at least one aromatic ring that contains a heteroatom ring member. Non-aromatic and/or carbocyclic rings may also be present in a heteroaryl group, provided that at least one ring is both aromatic and contains a ring member that is a heteroatom.

As used herein, a “heteroalkyl” group is an alkyl group that comprises at least one heteroatom covalently bonded to one or more carbon atoms of the alkyl group. Each heteroatom is independently chosen from N, O, S, Si, or P.

As used herein, “heteroarylalkyl” means a heteroaryl group linked via an alkylene moiety.

As used herein, “heteroarylene” means a divalent radical formed by the removal of two hydrogen atoms from one or more rings of a heteroaryl moiety, wherein the hydrogen atoms may be removed from the same or different rings (preferably the same ring), each of which rings may be aromatic or nonaromatic.

As used herein, “independently” indicates that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, “isolated” refers to a composition that includes at least 85% to 90% by weight, specifically 95% to 98% by weight, and even more specifically, 99% to 100% by weight of a particular compound in the composition, the remainder comprising other chemical compounds.

As used herein, “substituted” means a compound or radical substituted with at least one (e.g., 1, 2, 3, 4, 5, 6 or more) substituents independently selected from a halide (e.g., F⁻, Cl⁻, Br⁻, I⁻), a hydroxyl, an alkoxy, a nitro, a cyano, an amino, an azido, an amidino, a hydrazino, a hydrazono, a carbonyl, a carbamyl, a thiol, a C1 to C6 alkoxycarbonyl, an ester, a carboxyl, or a salt thereof, sulfonic acid or a salt thereof, phosphoric acid or a salt thereof, a C₁ to C₂₀ alkyl, a C₂ to C₁₆ alkynyl, a C₆ to C₂₀ aryl, a C₇ to C₁₃ arylalkyl, a C₁ to C₄ oxyalkyl, a C₁ to C₂₀ heteroalkyl, a C₃ to C₂₀ heteroaryl (i.e., a group that comprises at least one aromatic ring, wherein at least one ring member is other than carbon), a C₃ to C₂₀ heteroarylalkyl, a C₃ to C₂₀ cycloalkyl, a C₃ to C₁₅ cycloalkenyl, a C₆ to C₁₅ cycloalkynyl, a C₅ to C₁₅ heterocycloalkyl, or a combination including at least one of the foregoing, instead of hydrogen, provided that the substituted atom's normal valence is not exceeded.

The probes and processes herein are further illustrated by the following examples, which are non-limiting.

### EXAMPLES

### Example 1

### Exemplary Materials

Exemplary extracellular analyte markers include fluorescently labeled antibodies to identify pluripotency and differentiation state of cells and tissues, for example, SSEA4, SSEA1, Tra-1-60, Tra-1-80, and many different CD molecules used in immunophenotyping. Many CD markers are also used in cancer research, such as CD34, CD45, etc. Other examples of extracellular targets include various integrin (e.g. alpha5beta1), growth factor receptors (e.g. EGFR), and other extracellular proteins.

Exemplary intracellular analyte targets include fluorescently labeled antibodies used in stem cell research and determination of differentiation, for example, nanog, Sox2, oct4. Other examples include markers for apoptosis (e.g. caspasesmitochondrial markers (e.g. caspases, Bcl-2, PARP, etc), nuclear markers, cytoskeletal markers (e.g. tubulin or actin markers), and many other intracellular proteins.

Exemplary reference, first calibration, or second calibration probes would be a fluorescently labeled maleimide, more specifically labeled with Alexa 488, Alexa 555, Alexa 667, Texas Red, DyLight 488, DyLight649, Cy3, or Cy5.

Exemplary fluorescent labels incorporated into probes include Hydroxycoumarin, Aminocoumarin, Methoxycoumarin, Cascade Blue, Pacific Blue, Pacific Orange, Lucifer yellow, NBD, R-Phycoerythrin (PE), PE-Cy5 conjugates, PE-Cy7 conjugates, Red 613, PerCP, TruRed, FluorX, Fluorescein, BODIPY-FL, TRITC, X-Rhodamine, Lissamine Rhodamine B, Texas Red, Allophycocyanin (APC), APC-Cy7 conjugates, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, DyLight 350, DyLight 405, DyLight 488, DyLight 549, DyLight 594, DyLight 633, DyLight 649, DyLight 680, DyLight 750, and DyLight 800.

Exemplary samples include stem cells, such as H9 embryonic or various induced pluripotent stem cell lines; cancer cell lines, fibroblast and other established cell lines, primarily cell lines purified from biopsy or other means, mixed tissue samples and biopsies, samples from whole blood, and the like.

### Example 2

### Protocol for Labeling Samples with Probes

Preparation of samples for labeling and fixation includes several steps.

Wash samples with fresh medium. A specific sample would be induced pluripotent stem cells (e.g., ND2 line) colonies growing on a Matrigel-coated substrate. For flow cytometry, labeling will occur in suspension following harvest of cells from a plate, typically done using enzymes (e.g., Trypsin), calcium chelators (e.g., EDTA), scraping, or other means.

Add fixation buffer, specifically 3.7% to 4% paraformaldehyde. Some fixatives, like ethanol, both fix and permeabilize cell membranes. Allow cells to fix at temperatures from 4° C. to 37° C., specifically at room temperature (25° C.). Allow cells to fix from 30 min to 24 hours, specifically from 1 to 6 hours, more specifically for 3 hours.

Wash samples, specifically with at least 2× serial addition then removal of buffer over sample.

Addition of analyte probe, if analyte probe is for extracellular labeling: Typically, extracellular probes are labeled prior to permeabilization, as permeabilization can disrupt the integrity of the target within the cell membrane. A specific example of an extracellular target is the SSEA4 antigen, which is a common marker for pluripotency in stem cell research. A specific analyte probe would be a fluorescently labeled anti-SSEA antibody, more specifically, A1exa488 anti-SSEA4.

Add analyte probe at a concentration, specifically 1 to 100 μg/ml, more specifically approximately 20 μg/m in buffer (e.g. PBS).

Allow reaction to proceed from 10 min to 24 h, specifically from 30 min to 6 h, more specifically for 1 h. Temperature of reaction is from 4° C. to 25° C., specifically at room temperature (25° C.).

Wash sample as before.

Permeabilization of samples prior to application of calibration probes:

If intracellular antibody is to be used it can be added separately or at the same time as the protein-labeling probe. Permeabilization can occur before or in the same solution as the labeling step.

Permeabilize cells, specifically with Triton-X 100, specifically at a dilution of 0.1% in PBS. Add antibody according to manufacturer specifications, specifically 1 to 100 μg/ml, more specifically approximately 20 μg/m in buffer (e.g. PBS). Allow labeling to proceed from 30 min to 24 h, specifically from 1 to 3 hours. Reaction temperature is from 4 to 25° C., specifically at room temperature (25° C.). Wash sample as before.

Addition of analyte probe if analyte probe is for intracellular labeling. The analyte probe labeling reaction can be carried out before, after, or in the same mixture with the first calibration probe. A specific example of an intracellular target is the Nanog antigen, which is a common marker for pluripotency in stem cell research. A specific analyte probe would be a fluorescently labeled anti-nanog antibody, more specifically, A1exa488 anti-nanog.

Add analyte probe at a concentration, specifically 1 to 100 μg/ml, more specifically 20 μg/ml in buffer (e.g. PBS). Allow reaction to proceed from 10 min to 24 h, specifically from 30 min to 6 h, more specifically for 1 h. Temperature of reaction is from 4 to 25° C., specifically at room temperature (25° C.). Wash sample as before

Labeling samples with reference probe or first or second calibration probe:

Stain sample with calibration probe for 30 min to 24 h, more specifically from 1h to 6 h, more specifically for 3 h. Calibration probe is specifically a fluorophore labeled maleimide, more specifically an Alexa-488 Maleimide (the reference probe), and TexasRed Maleimide or a Cy5 Maleimide (the first or second calibration probe). Maleimide concentrations typically range from 0.1 to 500 μM. A number of concentrations over this range are selected to determine the maximum saturating fluorescence intensity or the concentration range that produces a fluorescence intensity that results in the most robust fluorescence output, i.e. an intensity that is least sensitive to fluctuations in fluorophore concentration used in labeling. It is estimated that such staining (for mammalian cells) will occur between concentration range of 20 to 200 μM. It is also suggested that the fluorescent maleimide be diluted with 95 to 99% unlabeled maleimide, for example, N-ethyl maleimide, such that total maleimide (unlabeled plus labeled) concentration falls within the specified concentration range. Allow reaction to proceed from 10 min to 24 h, specifically from 30 min to 3 hours. Temperature of reaction is from 4 to 25° C., specifically at room temperature (25° C.). Wash samples, as before.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix “(s)” as used herein is intended to include both the singular and the plural of the term that it modifies, thereby including at least one of that term (e.g., the colorant(s) includes at least one colorants). “Optional” or “optionally” means that the subsequently described event or circumstance can or cannot occur, and that the description includes instances where the event occurs and instances where it does not. As used herein, “combination” is inclusive of blends, mixtures, alloys, reaction products, and the like.

As used herein, “a combination thereof” refers to a combination comprising at least one of the named constituents, components, compounds, or elements, optionally together with one or more of the same class of constituents, components, compounds, or elements.

All references are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. “Or” means “and/or.” It should further be noted that the terms “first,” “second,” “primary,” “secondary,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity). The conjunction “or” is used to link objects of a list or alternatives and is not disjunctive; rather the elements can be used separately or can be combined together under appropriate circumstances.

What is claimed is:

1. A kit comprising:
a reference probe to react with a first sample; and
an analyte probe to combine with a second sample, a marker and a spectral probe to react to form the analyte probe to combine with the second sample, the marker to react with the reference probe to form the analyte probe to combine with the second sample, or a combination comprising at least one of the foregoing.

2. The kit of claim 1, further comprising a calibration probe to react with:
the second sample,
a third sample, or
a combination comprising at least one of the foregoing.

3. The kit of claim 2, further comprising a permeation agent.

4. The kit of claim 2, further comprising a reducing agent.

5. A process for staining, the process comprising:
forming a reference composition by reacting a reference probe and a first sample to form a reference; and
forming an analyte composition by combining an analyte probe and a second sample to form an analyte.

6. The process of claim 5, further comprising introducing a first calibration probe into the analyte composition.

7. The process of claim 6, further comprising forming a first calibrant by reacting the first calibration probe and the second sample.

8. The process of claim 7, further comprising forming a calibration composition by reacting a second calibration probe and a third sample to form a second calibrant.

9. A process for obtaining a normalized signal from an analyte, the process comprising:
combining a reference probe and a first sample to produce a reference composition;
reacting the reference probe and the first sample to form a reference in the reference composition;
determining a reference signal from the reference in response to subjecting the reference to radiation;
combining an analyte probe and a second sample to produce an analyte composition;
contacting the analyte probe and the second sample to form an analyte in the second composition;
determining an analyte signal from the analyte in response to subjecting the analyte to radiation; and
obtaining the normalized signal by normalizing the analyte signal to the reference signal.

10. The process of claim 9, further comprising introducing a first calibration probe in the analyte composition.

11. The process of claim 10, further comprising reacting the first calibration probe and the second sample to form a first calibrant in the analyte composition.

12. The process of claim 11, further comprising determining a first calibration signal from the first calibrant in response to subjecting the first calibrant to radiation.

13. The process of claim 12, further comprising combining a second calibration probe and a third sample to produce a calibration composition.

14. The process of claim 13, further comprising reacting the second calibration probe and the third sample to form a second calibrant in the calibration composition.

15. The process of claim 14, further comprising determining a second calibration signal from the second calibrant in response to subjecting the second calibrant to radiation.

16. The process of claim 15, wherein obtaining the normalized signal further comprises calibrating the analyte signal to the first calibration signal and the second calibration signal.

17. The process of claim 16, further comprising forming the analyte probe by reacting a marker and a reference probe.

18. The process of claim 9, wherein the reference probe comprises:
Q1-L1-A1;
Q1-A1;
Q1-R; or
a combination comprising at least one of the foregoing,
wherein Q1 is an amine reactive moiety, a thiol reactive moiety, or combination comprising at least one of the foregoing;
L1 is a linker group; and
A1 is a spectrally active group; and
R is a terminal group.

19. The process of claim 9, wherein the analyte probe comprises:
M-Q′-L1-A1;
M-Q′-A1;
M-Q′-A1-R;
M-A1;
M-A1-R;
M-L1-A1;
M-L1-A1-R; or
a combination comprising at least one of the foregoing,
wherein M is a marker;
Q′ is a product moiety;
L1 is a linker group;
A1 is a spectrally active group; and
R is a terminal group.

20. The process of claim 13, wherein the first calibration probe and the second calibration probe independently comprise:
Q2-L2-A2;
Q2-A2;
Q2-R;
or a combination comprising at least one of the foregoing,
wherein Q2 is an amine reactive moiety, a thiol reactive moiety, or combination comprising at least one of the foregoing;
L2 is a linker group; and
A2 is a spectrally active group; and
R is a terminal group.
